(19) United States
(12) Patent Application Publication
(10) Pub. No.: US 2007/0087330 A1
(43) Pub. Date: Apr. 19, 2007

(54) Viral deconstruction through capsid assembly in vitro

(76) Inventors: Jairam R Lingappa, Seattle, WA (US); Jairam R. Lingappa, Atlanta, GA (US); Vishwanath R. Lingappa, San Francisco, CA (US)

Correspondence Address: JONATHAN ALAN QUINE;THE QUINE INTELLECTUAL PROPERTY LAW GROUP, P.C., 2033 CLEMENT AVE. #200, ALAMEDA, CA, 94501, US

(21) Appl. No.: 10/527,973

(22) PCT Filed: Sep. 11, 2003
(86) PCT No.: PCT/US03/28622

§ 371 Date: Aug. 2, 2006

Related U.S. Application Data
(60) Provisional application No. 60/410,572, filed on Sep. 13, 2002.

(51) Int. Cl. C12Q 1/70; C12Q 1/68; C07H 21/04; C12P 21/06; C40B 30/06; C07K 14/005; C07K 16/10

(52) U.S. Cl. 435/5; 435/6; 435/69.1; 435/456; 435/325; 530/350; 530/388.3; 536/23.72; 977/802

(57) ABSTRACT

A cell-free method for translation and assembly of viral capsid and capsid intermediates is disclosed for use in deconstructing an unknown virus and for screening for compounds that inhibit assembly of viral capsids for the unknown virus.

### INTRODUCTION

1. Field of the Invention

The invention is concerned with methods and compositions for identifying drug targets for inhibiting viral replication and methods and/or compositons for preventing and/or treating infection by an unknown and/or synthetic virus, particularly a virus used as a bioweapon.

2. Background of the Invention

Biological warfare can be used to decimate human populations and to destroy livestock and crops of economic significance. Recent terrorist attacks in the U.S. and elsewhere have brought into focus the threat posed by biological weapons and have provoked discussion of mass vaccination strategies for both military personnel and civilian populations. The strategies assume the use of classical bioweapons agents. However, the power of genetic engineering raises the possibility of advanced-generation bioweapons agents that are even more virulent than their naturally occurring counterparts and that are capable of evading current vaccine defenses.

The list of classicsal biological agents that could be used as bioweapons includes over 100 bacteria, viruses, rickettsia, fungi, and toxins. However, most experts believe that the most likely bioweapons include anthrax, smallpox, plague, botulinum toxin, tularemia, and viral hemorrhagic fevers. Using bioengineering of these materials, artificial viruses, antibiotic resistant strains of microorganisms, toxins and other exotic bioweapons such as bacterial proviruses (viruses inserted into bacteria, so that when a person is treated for the bacterial illness with antibiotics, the virus is released) can be created.

In the group of hemorrhagic fever viruses that are most likely to be used as bioweapons are Ebola, Marburg, Lassa Fever, New World Arenavirus, Rift Valley fever, yellow fever, Ornsk hemorrhagic fever, and Kyasanur Forest Disease. Like smallpox and anthrax, the Centers for Disease Control and Prevention (CDC) considers hemorrhagic fever viruses “category A” biological weapons agents, because they have the potential to cause widespread illness and death, and would require special public health preparedness measures to contain an outbreak. Ebola and Marburg, which belong to the Filoviridae family of viruses, can be spread from person to person and are among the most deadly hemorrhagic fever illnesses. Ebola kills 50 to 90 percent of those infected, while Marburg is fatal 23 to 70 percent of the time. There are no specific treatments for an outbreak of these viruses. Each of the above viruses is considered to be a candidate for use by bioterriorists because of its virulence, stability in the environment, high infectivity, and in some cases high degree of communicability.

If an attack were to occur using a virus as a bioweapon, diagnosing the causative agent so as to determine the appropriate treatment, whether a hemorrhagic fever virus or other virus, may be difficult. As an example, most hemorrhagic fever illnesses begin with a fever and rash, which is similar to other more common illnesses. Not only are most clinicians not familiar with these diseases, there are no widely available diagnostic tests and special facilities are required for working with these viruses. In the US, the CDC in Atlanta, Ga. and USAMRIID in Frederick, Md. house the only facilities equipped to diagnose hemorrhagic fever viruses. For known viruses such as Ebola, antigen-capture enzyme-linked immunosorbent assay (ELISA) testing, IgM ELISA, polymerase chain reaction (PCR), and virus isolation can be used to establish a diagnosis within a few days of the onset of symptoms. Persons tested later in the course of the disease or after recovery can be tested for IgM and IgG antibodies; the disease can also be diagnosed retrospectively in deceased patients by using immunohistochemistry testing, virus isolation, or PCR. These tests not only potentially expose laboratory staff to infection, but also require knowledge of the causative agent. Even with this knowledge, the availability of antibodies that react with the causative agent, the availability of appropriate primers for PCR and the ability to grow sufficient virus in appropriate living cells for virus isolation may be lacking. If the virus has mutated, has been genetically altered and/or is a hybrid virus, available antibodies and primers may no longer be useful for diagnosis, and without information as to the nature of the virus, it may be difficult to determine appropriate host cells for growing the virus for isolation for diagnosis and potential vaccine development and for determining an appropriate treatment regimen.

For treatment, few effective therapies or vaccines are available to deal with viruses in general and hemorrhagic fever viruses in particular. The antiviral drug ribavirin is recommended only for the treatment of the Arenaviridae and the Bunyaviridae families of viruses. For the Filoviridae (Ebola, Marburg) and the Flaviviridae, currently supportive care only is available to treat the symptoms of infected patients. There is a vaccine to prevent yellow fever, but it is not widely available and it would not be useful to provide protection after exposure. Moreover, the most threatening engineered pathogens of the bioweapons arsenal may remain unknown until they are used in an attack. It therefore is of interest to develop methods and compositions for identifying potential drug targets and methods and compositions for preventing and/or treating infection with unknown viruses such as those used as bioweapons and to develop methods and compositions for delivering productive antibodies to those who are potential targets of bioterrorism. There also is a need for compounds for treatment of infected individuals that specifically inhibit viral replication even in the absence of precise knowledge concerning the infective agent.

### RELEVANT LITERATURE

Cell free systems have been used to study the assembly of viruses that preform into capsids in the cytoplasm (Lingappa et al (1994) J. Cell Biol. 125: 99-111; Sakalian et al (1996) J. Virol 70: 3706-15; and Sakalian and Hunter (1999) J. Virol 73: 8073-82) as well as those that assemble at membrane interfaces (Lingappa et al (1997) J. Cell Biol 136: 567-81; Singh et al (2001) Virology 279: 257-70) and Zimmerman et al (2002) Nature 24: 88-92. However, assembly intermediates and host proteins involved in capsid formation either were not examined in these studies, and/or their potential use in identifying unknown viruses and/or treatment and prevention of infection with unknown viruses was not recognized.

### SUMMARY OF THE INVENTION

This invention relates to methods and compositions for identifying and isolating viral and host proteins involved in capsid assembly, particularly of an unknown or a non-naturally occurring virus, using a cell-free translation system and to methods and compositions for identifying drugs that specifically target the identified host and viral proteins and inhibit capsid assembly. The method for identifying the host and viral proteins includes the steps of identifying viral nucleic acid encoding capsid protein(s), preparing a transcript in vitro from the viral nucleic acid so identified, translating the viral transcript to produce transcription products in a cell-free protein translation mixture that contains any necessary host proteins (chaperones) for capsid assembly; incubating the resulting mixture for a time sufficient to synthesize viral capsid assembly proteins and assemble the newly synthesized proteins into capsid assembly intermediates, isolating the capsid assembly intermediates, and separating the capsid assembly intermediates into their component viral encoded proteins and host proteins. Methods for identifying an agent for treating symptoms of infection with an unknown viral agent include high thoroughput screening of potential small molecules using the cell-free expression system and comparing the amount of capsid formed in the presence of a test compound with capsid assembly in the absence of a test compound. An alternative method is to compare one or more biochemical characteristic of the host proteins to the biochemical properties of individual members of a host protein library that includes biochemical characteristics of a plurality of viral capsid assembly chaperones individually cross-referenced with one or more small molecules that inhibit interaction between an individual member of the library and a viral capsid protein and providing an animal subject to infection or infected with the unknown virus with a small molecule that is cross-referenced with an individual member of the library that has one or more biochemical characteristic in common with the host protein. If the virus is a naturally occurring virus, or is a hybrid relatd to a naturally occurring virus, identifying the host protein in the library can be used to identify the unknown virus. The invention finds use in identifying compounds that specifically inhibit the interaction of viral and host proteins that are involved in capsid formation and thereby inhibit viral replication and can be used in viral prevention and treatment protocols. The invention also finds use in the preparation of antibodies to the viral capsid proteins, the assembly intermediates, and the host proteins or their conformers involved in capsid assembly, for diagnosis and vaccines.

### BRIEF DESCRIPTION OF THE FIGURES

FIG. 1 shows a diagram of a cell-free system for viral capsid assembly. Capsid transcript is synthesized in vitro and added to wheat germ extract, an energy regenerating system, 19 unlabeled amino acids, and one labeled amino acid (typically ³⁵S-met or ³⁵S-cys). Reactions are incubated at 26° C. for 150 min. Translation of capsid proteins is followed by a series of post-translational events (that differ for various types of viral capsids), resulting in 20-40% of capsid chains forming completely assembled capsids. At the end of the reaction, products of different sizes (i.e. unassembled, partially- assembled, and completely-assembled core polypeptides) can be separated from each other by velocity sedimentation on sucrose gradients.

FIG. 2 shows migration of HIV capsids formed in a cell free system (FIG. 2A) and in a cellular system (FIG. 2B) on velocity sedimentation gradients, in the form of plots of the buoyant density of each of the sequential fractions collected, assessed by refractive index (open circles), and of the amount of Gag protein in each fraction, as assessed by densitometry (closed circles).

FIG. 3 shows pulse-chase analysis of HIV capsid assembly by velocity sedimentation in a continuously labeled cell-free reaction mixture (FIG. 3A) where the calculated positions of 10S, 80S, 150S, 500S, and 750S complexes are indicated by markers at the top of the graph, and in reactions to which unlabeled ³⁵S cysteine was added 4 minutes into the reaction and aliquots were taken for sedimentation analysis after 25 minutes (FIG. 3B) and 15 minutes of reaction (FIG. 3C), and samples were further analyzed by SDS gel and radiography.

FIG. 4. A 68 kD host protein selectively associates with HIV-1 Gag in the cell-free system.

(A) Cell-free translations were programmed with transcripts for either HIV-1 Gag, β-tubulin, α-globin, HBV core, or the assembly-defective p41 mutant in HIV-1 Gag^{7,11,15} Reaction products were subjected to immunoprecipitation under native conditions usin the 23c monoclonal antibody (23c) or non-immune rat IgG (N), as described previously¹⁵. Autoradiograph of immunoprecipitated samples is shown. The total lane (T) in each set shows 5% of the input translation product.

(B) A cell-free assembly reaction programmed with HIV-1 Gag transcript was immunoprecipitated under either native conditions or after denaturation as indicated using the antibodies described in (A). The total lane (T) shows 5% of the input translation product.

(C) Antibody to 23c was pre-incubated with different amounts of fractionated WG supernatant (containing soluble proteins of 40 S or less) before incubation with a 2 μl cell-free reaction programmed with HIV-1 Gag transcript. Immunoprecipitations wee performed under native conditions. Account of WG extract present in a 2 μl cell-free reaction was defined as one WG equivalent. The amount of WG supernatant used for pre-incubating the antibody ranged from 2 to 200 WG equivalents. (100 WG equivalents represents a final WG protein concentration of 14 mg/mi.) The graph shows the relative amount of radiolabeled Gag that was immunoprecipitated (in arbitrary units), as determined by densimetry of Autoradiographs. Bars indicate standard error of the mean from 3 independent experiments. Inset shows a representative autoradiograph of the immunopreceipitations, with amount of WG equivalents added during pre-incubation indicated above.

(D) A high-speed supernatant of WG extract was analyzed directly by Western blotting using the 23c antibody (lane2), or was first subjected to immunoprecipitation under native conditions using either non-immune rat IgG (lane 1) or the 23c antiody (lane 3) and then analyzed by immunoblotting with the 23c antibody. The filled arrow indicates the 68 kD antigen in WG extract that is recognized by the 23c antibody upon direct Western blotting (lane 2) or upon immunoprecipitation with 23c antibody followed by Western blotting (lane 3). Secondary antibody used for immunoblotting was Protein G coupled to HRP, which also recognizes the heavy and light chains of antibodies used for immunoprecipitation as indicated (HC and LC). (Note that HC and LC chains of different antibodies used in lanes 1 and 3 migrate differently.) Molecular weight markers are indicated to the left, and antibodies used for immunoprecipitation (IP) and Western blotting (WB) are indicated above each lane.

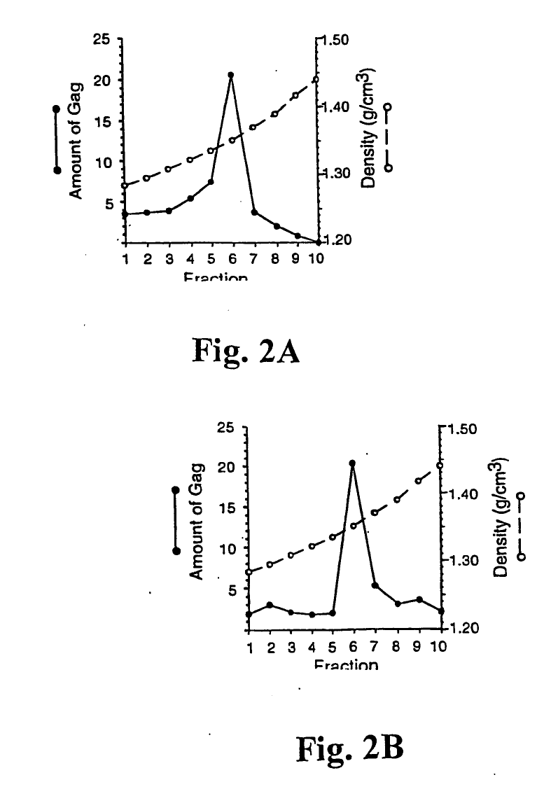

FIG. 5. HP68 associates with HIV-1 capsid assembly intermediates. (A) Cell-free assembly reactions were programmed with HIV-1 Gag transcript as in FIG. 2, except that reactions contained ³⁵S-cysteine^{7,15}. Three minutes into the translation, excess unlabeled cystein was added to eliminate further radiolabeling, and aliquots of the translation were removed for analysis at various times, as indicated (chase time). These were analyzed directly by SDS-PAGE and AR to determine the total amount of radiolabeled Gag present at each time, and by inununoprecipitation under native conditions with either 23c or non-immune rat IgG (data not shown). To determine relative 23c immunoreactivity shown in (A), autoradiographs of immunoprecipitated samples from 3 independent experiments were quantitated by densitometry, normalized to total radiolabeled Gag synthesis for each time point, averaged, and then graphed with respect to chase time. Error bars indicate standard error of the mean. (B, C) Continuously labeled cell-free translations were programmed with Gag transcript, incubated for 2 hours, and then subjected to velocity sedimentation on 13 ml sucrose gradients, as described in Methods. Total amount of radiolabeled Gag present in each fraction was quantitated and graphed (B). Calculated positions for complexes of various S values are shown above. Dark bar indicates the migration position of authentic fully assembled immature HIV-1 capsids on a parallel velocity sedimentation gradient (as determined by comparison to authentic immature capsids. Arrows indicate the positions of previously described capsid assembly intermediates. Each gradient fraction was also subjected to immunoprecipitation under native conditions using the 23c antibody and analyzed by SDS-PAGE and AR. Amount of radiolabeled Gag co-immunopreceiptated by the 23c antibody was antitated and graphed using arbitrary units (C). S value markers and dark bar are described in A above. No radiolabeled Gag polypeptides were immunopreceipitated by non-immune serium from any of the fractions (data not shown). This experiment was repeated in triplicate; data shown is from one representative experiment.

FIG. 6. Amino acid sequence of WGHP68. Alignment of WGHP68 with HuHP68, previously termed RNase L inhibitor, reveals an overall amino acid identity of 71%. Gaps in alignment are indicated by dashes, identical amino acids by asterists, and conserved amino acids by dots. Open boxes indicate the two P-loop motifs present in both homologues. Black boxes indicate wo regions of amino acid sequence that were obtained by microsequencing and used to construct degenerate oligonucleotides for PCR. The arrow indicates the last amino acid in the N-terminal truncation mutant WGHP68-Tr1.

FIG. 7 shows truncated HP68 blocks virion production. (FIGS. 7A-D), Cos-1 (FIGS. 7A, B) or 293T (FIGS. 7C, B) cells co-transfected with varying amounts of plasmid expressing WGHP68-Tr1 and empty vector, as indicated, plus plasmids for expression of HIV-1 Gag (FIGS. 7A, B) or pBRUΔenv (FIGS. 7C, B). Medium (FIGS. 7A, C) was immunoblotted with Gag antibody (p55; p24), and reprobed with antibody to light chain tracer (LC). Cell lysates (FIGS. 7B, D) were immunoblotted using WGHP68 antiserum (HP) or Gag antibody (p55; p24), and reprobed using actin antibody (actin). Arrows: open, native HP68; filled, WGHP68-Tr1. Bar graphs: blots from 3 experiments quantitated using sample dilution standard curves.

FIG. 8 shows HuHP68 co-immunoprecipitates HIV-1 Gag in mammalian cells. Native (NATIVE) or denaturing (DENAT) immunoprecipitations using αHuHP68b (HP) or non-immune serum (N), followed by immunoblotting (IB) with antibody to HuHP68 (IB: HP) or Gag (IB: Gag), were performed on: (FIG. 8A) 293T cells transfected with pBRUΔenv, ±RNase A treatment; (FIG. 8B), Cos-1 cells expressing Gag; (FIG. 8C), Cos-1 cells expressing Gag (Gag), an assembly-incompetent Gag mutant (p41), an assembly-competent Gag mutant (p46), or control vector (native immunoprecipitation only); or (FIG. 8D), chronically HIV-1-infected ACH-2 cells. HIV-1 p24 and p55 (arrows), 5% input cell lysate (T), and 10 μl medium (T medium) are indicated.

FIG. 9 shows HuHP68 co-immunoprecipitates HIV-1 Gag and Vif but not Nef or RNase L. (FIG. 9A), Cos-1 cells transfected with pBRUΔenv or HIV-1 Gag plasmids were immunoprecipitated under native (NATIVE) or denaturing (DENAT) conditions using αHuHP68b (HP) or non-immune serum (N), and immunoblotted (IB) with antibody to HuHP68 (HP), HIV-1 Gag, HIV-1 Vif, HIV-1 Nef, RNase L (RL), or Actin. Total (T): 5% of input cell lysate used in immunoprecipitation (HP: 10%). Top of some actin lanes contains heavy chain cross-reacting to secondary. (FIG. 9B) shows the results with lysates of pBRUΔenv-transfected Cos-1 cells, harvested in 10 mM EDTA-containing buffer, and co-immunoprecipitated using beads pre-incubated with HuHP68 peptide or diluent control.

FIG. 10. HP68 is recruited by HIV-1 Gag in mammalian cells. Cos-1 cells were transfected with pBRUΔenv (columns 1-3) or pBRUp41Δenv, which enclodes a stop codon after residue 361 in Gag (column 4) and examined by double-label indirect immunofluorescence. Fields were examined for HP68 staining (red, shown in top row), or Gag staining (green, middle row). Images were merged showing overlap of HP68 and Gag (yellow; bottom row). Bar in lower left corresponds to approximately 50 μm.

FIG. 11. HuHP68 co-immunoprecipitates HIV-1 Vif but not RNase L in mammalian cells. (A) Cos-1 cells ransfected with either pBRUΔenv or Gag expression plasmids were harvested and subjected to immunoprecipitation under native conditions (NATIVE) or after denaturation (DENAT) using αHuHP68b (HP) or non-immune serum (N), and analyzed by immonoblotting (IB) with antibody to either HuHP68 (HP), HIV-1 p55 Gag, HIV-1 Vif, HIV-1 Nef, RNase L (RL), or Actin as indicated. Total lane (T) shows 5% of the input cell lysate used for immunoprecipitation. When antibodies enerated in rabbits are used for immunoblotting (HP, RL, and Actin immunoblots), a heavy chain artifact can be seen at 50 kD in IP lanes (most prominent in actin panel). (B) Cos-1 cells ransfected with bPruΔenv were also subjected to immunoprecipitation under native conditions in the presence of 10 mM EDTA, and in the presence or absence of the HuHP68 peptide (200 μM) that was used to generate αHuHP68 antiserum (HP Peptide+or −). DMSO alone (0.25%), which was used to dissolve the peptide, bad no effect on co-immunoprecipitation of Gag and Vif by αHuHP68 (data not shown). Total lane (T) shows 5% of the input cell lysate used for immunoprecipitation, except for the HP immunoblot total which represents 10% of input cell lysate. All experiments were performed 3 times and data shown are from a representative experiment.

FIG. 12 shows that in Cos-1 cells, HP68 is associated with HIV-1 and HIV-2 Gag from two primary isolates, but not with a mutant of HIV-1 Gag or HIV-2 Gag truncated at the CA/NC junction. Cos-1 cells were transfected with plasmids encoding HIV-1 Gag, or Gag from two different primary isolates of HIV-2 (506 and 304), SIVmac239 or versions of HIV-1, HIV-2, or SIV Gag that are truncated at the CA/NC junction (Tr). Lysates were subjected to immunoprecipitation with affinity-purified antibody to EP68 (HP) or non-immune serum (N) under either native or denaturing conditions, as indicated, and analyzed by immunoblotting (IB) with antibody to either HP68 (HP) or antibody to Gag. Total (Tot.) shows 5% pf immunoprecipitation input HIV-2 primary isolate cDNAs were obtained from Dr. S. L. Hu; and SIVmac239 cDNA was obtained from Dr. P. Luciw.

FIG. 13 shows velocity sedimentation of HCV and HBV core assembled in a cell-free system. Cell-free reactions programmed with HCV or HBV core transcript were incubated for 2.5 h and analyzed by velocity sedimentation on 2 ml sucrose gradients containing 1% NP40 (55,000 rpm×60 min. in Beckman TLS55 rotor). Fractions (200 microliters each) were collected from top of gradient and examined by SDS-PAGE and autoradiography. In both reactions, core chains form 100S particles and complexes of other sizes.

FIG. 14 shows that 100S particles produced in the cell-free system have the buoyant density expected for HCV capsides. Products of a cell-free assembly reaction programmed with HCV core transcript were separated by velocity sedimentation, as in FIG. 13. Fractions 6 and 7 (100 S core particle) were analyzed by equilibrium centrifugation (50,000 rpm×20 hours using a TLS55 Beckman rotor) using a 337 mg/ml CsC1 solution. Fractions were collected, TCA precipitated, analyzed by SDS-PAGE and autoradiography, and quantitated by densitometry. HCV core protein peaked in fraction 6. The density of fraction ⅚ (middle of the gradient, indicated with arrow) is 1.25 g/ml.

FIG. 15 shows mutants containing the hydrophilic interaction domain of core assemble in the cell-free system. Cell-free reactions were programmed with wild-type HCV core (C191) or mutants in core truncated at amino acids 122 or 115 (C122 vs. C115), and analyzed by velocity sedimentation on 2 ml sucrose gradients (as described in FIG. 13). Fractions were examined by SDS-PAGE, and autoradiographs were quantitated. Graph shows amount of each core protein present in 100S particles as % of total synthesis.

FIG. 16 shows the strategy for co-immunoprecipitation of HCV core.

FIG. 17 shows co-immunoprecipitation of HCV core by 60-C anti-serum. Cell-free reactions were programmed with either HCV core, HIV-1 Gag, or HBV Core. During assembly, reactions were subjected to immunoprecipitation (IP) under native conditions with antisera directed against different epitopes of TCP-1 (60-C, 60-N, 23c, and 91a) or with non-immune serum (NI). IP eluates were analyzed by SDS-PAGE and autoradiography. Tot shows a 5% of the input used to program the IP. Arrows show positions of full-length capsid proteins.

FIG. 18 shows sucrose gradient fractionation of HBV core cell-free translation products. HBV core cDNA was transcribed arid translated for 120 min. The translation products were then layered onto a 2.0-ml 10-50% sucrose gradient and centrifuiged at 200,000 g for 1h. 200-microliter fractions were removed sequentially from top to bottom of the gradient (lanes 1-11, respectively) and the pellet (lane 12) was resuspended in 1% NP-40 buffer. Aliquots of each fraction were analyzed by SDS-PAGE and autoradiography to detect the radiolabeled 21-kD core polypeptide band. Two minor HBV core bands of lower molecular weight are seen (in both in vitro translations as well as in core protein produced by transfecting E. coli). These are thought to be either degradative products or the result of initiation of translation at internal methionines. Positions of molecular weight standards are shown. The position of catalase, an 11-S standard, in this type of gradient (as determined by Coomassie staining) is shown with an arrow. Likewise the migration of recombinant core particles, known to have a sedimentation coefficient of ˜100S, is shown with an arrow. Radiolabeled HBV core polypeptides migrate in three regions of this gradient: top (T) corresponding to fractions 1 and 2; middle (M) corresponding to fractions 6 and 7; and pellet (P) corresponding to fraction 12, as shown with dark bars.

FIG. 19 shows pulse-chase analysis of assembly of HBV core particles. In vitro transcription and translation were performed with an initial 10-min pulse of [35S] cysteine followed by a chase with unlabeled cysteine for either 10 (A),35 (B), 50 (C), or 170 min (D). Translation products were layered on sucrose gradients, centrifuged, fractionated, and analyzed by SDS-PAGE and autoradiographed as previously described. Autoradiographs are shown to the right of the respective bar graphs that quantitate density of bands present in the top (T), middle (M), and pellet (P) of the respective autoradiographs. The total amount of radiolabeled full-length core polypeptide present at each time point is the same, as determined by quantitation of band densities of 1-microliter aliquots of total translation. Labeled core polypeptides chase from the top to the pellet and finally to the middle of the gradient over time.

FIG. 20 shows preparation and characterization of a polyclonal antiserum against a cytosolic chaperonin. A, shows alignment of an amino acid sequence present within mouse TCP-1 (positions 42-57) (Lewis et al. 1992 Nature 358:249-252), S. shibatae TF55 (a heat shock protein of a thermophilic archaebacterium) (positions 55-70) (Trent et al. 1991 Nature 354:490-493) and yeast TCP-1 (positions 50-65) (Ursic and Culbertson, 1991 Mol. Cell Biol. 11:2629-2640). Amino acids identical to those in the mouse sequence are designated by (.). A synthetic peptide was synthesized corresponding to amino acids 42-57 from mouse TCP-1 because of the high degree of homology in this region. This peptide was conjugated to carrier protein or cross-linked to itself and used to generate rabbit polyclonal antisera (anti 60). Immunoprecipitations were performed with this antiserum under denaturing conditions on whole cell extracts of steady state, [35S]methionine-labeled HeLa cells. A protein of ˜60 kD was precipitated by anti 60, shown in B, lane 1. As a control, B, lane 2 shows an immunoprecipitation under denaturing conditions done with antiserum to hsp 70 in the same experiment. Molecular weight markers (92, 68, and 45 kD) are indicated to the left with open arrowheads. Under native conditions, anti 60 also immunoprecipitates a 60-kD protein in solubilized HeLa cells. To further characterize the antigen recognized by this antiserum, rabbit reticulocyte extract and wheat germ extract were layered onto 10-50% sucrose gradients, centrifuged at 55,000 rpm for 60 min in a TL-100 Beckman ultracentirfuge, fractionated, and analyzed by SDS-PAGE. The proteins were transferred to nitrocellulose and were immunoblotted with anti 60 as shown in C. To determine S values, protein standards were centrifuged in a separate gradient tube at the same time and fractions were visualized by Coomassie staining of SDS-PAGE gels. The positions of these markers (BSA and α-macroglobulin) are indicated with arrows. Molecular weight markers (68 and 45 kD) are indicated to the right with open arrowheads. In both immunoblots, only a single band was recognized, representing a 60-kD protein, migrating in the 20-S position. Thus, anti 60 appears to recognize a 60-kD protein (CC 60) that migrates in the 20-S region and is likely to be either TCP-1 or homolog.

FIG. 21 shows immunoprecipitation of HBV core translation products. HBV core was translated in vitro for 60 min. Translation products were centrifuged on sucrose gradients and fractionated. Fractions from the top (“T”), middle (M) and pellet (P) regions were divided into equal aliquots and immunoprecipitations were performed as described in Materials and Methods under either native (A) or denaturing (B) conditions using either anti-core antiserum (C), nonimmune serum (N), or anti 60 (60). Immunoprecipitated labeled core protein was visualized by SDS-PAGE and autoradiography C shows a separate experiment in which native immunoprecipitations were performed on HBV core translation products following equilibrium density centrifugation. In this experiment, HBV core was translated for 150 min and centrifuged on sucrose gradients as described. Material from the middle (lanes 6 and 7) of sucrose gradients was pooled and centrifuged on CsC1 equilibrium gradients: Fractions 3 and 6-were collected, divided into equal aliquots and immunoprecipitated under native conditions using either anti-core antiserum (C), nonimmune serum (N) or anti 60 (60). Exposure times for autoradiographs were identical for each of the three lanes (C, N; and 60) within a set, but vary between sets.

FIG. 22 shows that unassembled core polypeptides can be chased into multimeric particles. HBV core transcript was diluted by 50% with mock transcript, and then translated for 120 min. Translation products were divided into three aliquots. One aliquot was put on ice (A). To a second aliquot was added a translation of HBV core polypeptides that was made using 100% transcript and only unlabeled amino acids that had been incubated for 45 min. This mixture was then further incubated for either 45 (B) or 120 min (C). To a third aliquots was added a translation of mock transcript that had been incubated for 45 min, and this mixture was further incubated for 120-min (D). All four samples were then centrifuged on sucrose gradients and fractions were removed and analyzed by SDS-PAGE and autoradiography as previously described. Unassembled core polypeptides shown in A are found to move first into the pellet and then into the middle over time (B and C, respectively) with the addition of high concentration of (unlabeled) HBV core polypeptide chains. In contrast, with addition of mock translation (D), core polypeptides remain at the top of the gradient.

FIG. 23 shows completed capsids are released from the isolated pellet. Following translation of HBV core transcript for 30 min, the translation product was diluted in 0.01% Nil(kol buffer and centrifuged on a 10-50% sucrose gradient. The supernatant was removed and the pellet was resuspended in buffer and divided into equal aliquots. To one aliquots was added apyrase (A, top) while the control was incubated in buffe alone (A, bottom). Incubations were done at 25° C. for 90 min. Reaction mixtures were then centrifuged on standard 10-50% sucrose gradients. Fractions were analyzed by SDS-PAGE and autoradiography. In a separate experiment (B) the pellet was isolated and resuspended in identical fashion. To one aliquot was added wheat germ extract as well as unlabeled energy mix. (B, top); to the second aliquot was added wheat germ extract and apyrase (B, botton). The reactions were incubated at 25° C. for 180 min and centrifuged as described for A. Treatment with apyrase (with or without wheat germ extract) resulted in release of radiolabeled material that migrated in the middle of the gradient. That this material represents complete capsids was confirmed by centrifugation on equilibrium CsC1 gradients along with authentic capsid as a control (data not shown). In contrast, treatment with wheat germ extract and energy mix resulted in generation of radiolabeled material that migrated in the top as well as the middle gradient. The material in the middle of these gradients was also shown to include completed capsids by centrifugation on CsC1 along with authentic capsids as a marker.

FIG. 24 shows electron micrographs of capsids produced in a cell-free system. Translation of HBV core transcript (Cell-Free) as well as translation of an unrelated protein (GRP-94 truncated at NcoI, referred to here as Control) were performed for 150 min and these products as well as recombinant capsids (authentic) were centrifuged to equilibrium on separate CsC1 gradients. Fraction 6 from each gradient was collected and further sedimented in an Airfuge. In single blinded fashion the pellet of each was collected, resuspended, and prepared for EM by negative staining. Identity of samples was correctly determined by the microscopist. No particles resembling capsids were seen in the control samples. Bar, 34 nm.

FIG. 25 shows that N-terminal deletion mutants of HCV core fail to assemble in a cell-free system.

### DETAILED DESCRIPTION OF THE INVENTION

The present invention uses a cell-free system for translation and assembly of viral capsids as a means of identifying potential drug targets for inhibiting viral replication and small molecules that interact with the drug targets for use in treating and/or preventing viral infection in a plant or an animal, particularly a mammal such as a livestock animal and more particularly a human, even if the viral agent is unknown and/or non-naturally occurring. This invention is based on the fact that all viruses contain a protein shell (capsid) surrounding a nucleic acid containing core (the complete protein-nucleic acid complex is the nucleocapsid) and the finding that all viruses examined to date in the cell-free system require one or more host protein or chaperone for capsid assembly. Previously it had been believed that some simple viruses formed spontaneously from their dissociated protein components while others required enzyme-catalyzed modifications of the capsomers to trigger assembly. However, in recent studies (see for example PCT/US98/02350) using a cell free translation system, it was shown that HIV capsid assembly proceeds through one or more capsid assembly intermediates. This finding has now been extended to other unrelated viruses including HCV and this information, together with information relating to HBV (Lingappa et al., J Cell Biol (1994) 125:99-111 ), now suggests that viral capsid structures in general are formed in an ordered sequence of assembly intermediates culminating in the final completed capsid structure. These assembly intermediates are complexes that include both virally encoded proteins and host proteins that act as chaperones. Therefore, although the capsids of many viruses differ in protein composition, a general viral pathway for capsid formation involving host proteins is now evident and can be used as a means to identify potential drug targets for unknown viral agents through screening of compounds that inhibit the capsid assembly process and/or isolation of the assembly intermediates that are observable during capsid assembly using a cell free system. Exemplified herein are host cell proteins, capsid assembly pathways and intermediates for three viruses, HIV, HBV, HCV. Although the viruses themselves are different and the host proteins identified as involved in capsid assembly are different, the pathways for capsid assembly are similar for these three viruses. Because the viruses studied are so dissimilar, it is expected that similar pathways are used for capsid assembly by other virues, and that the intermediates described herein have analogous counterparts in such other capsid assembly pathways. These counterparts can be identified using the general manipulations described below even if the virus itself is unknown.

In the method for identifying potential drug targets, viral nucleic acid from an unknown viral agent is screened to identify nucleic acid encoding a viral capsid gene for example by sequence homology to known capsid genes. The nucleic acid is then used prepare a transcript in vitro which in turn is used to program a cell free translation system for preparation of viral capsids; formation of capsids is evidence that the identified nucleic acid is required for capsid assembly. Capsid assembly intermediates and trans acting host proteins involved in the capsid assembly pathway are isolated and sequenced and then used in screening for antiviral compounds that inhibit the interaction of the identified host proteins and virally-encoded capsid proteins, for example using the cell free translation system. The phrase “capsid assembly pathway” refers to the ordered set of serial assembly intermediates required for formation of the final completed capsid structure. To progress from one assembly intermediate to the next, a specific modification or modifications of the intermediate fake place. The phrase “cell-free translation” refers to protein synthesis carried out in vitro in a cell extract that is essentially free of whole cells. The phrase “cell-free translation mixture” or “cell-free translation system” refers to a cell-free extract that generally includes sufficient cellular machinery and components to support protein translation including transfer RNA, ribosomes, a full complement of at least 20 different amino acids, an energy source, which may be ATP and/or GTP, and an energy regenerating system, such as creatine phosphate and creatine phosphokinase. Alternatively, antiviral compounds for treatment of a viral infection can be identified by isolating the capsid assembly intermediates such as by denaturing the complexes and separating them into their component viral encoded proteins and host proteins. One or more biochemical characteristic of the host protein, such as the amino acid sequence of the region of the host protein that binds to the viral capsid protein or the identification of antibodies that bind to this region is compared to a library that includes biochemical characteristics of a plurality of viral capsid assembly chaperones individually cross-referenced with one or more small molecules that inhibit interaction between an individual member of the library and a viral capsid protein. A small molecule that is cross-referenced with an individual member of the library that has a biochemical characteristic in common with the host protein can then be used as a treatment for symptoms associated with infection with the unknown agent or to prevent infection with the unknown agent.

The subject invention offers several advantages over existing technology. A major advantage is that in the cell-free system the universal step in the lifecycle of all viruses, formation of the capsid, can be broken down to enable isolation of assembly intermediates that are uniquely associated with each class of viruses and identification of one or more distinct host factors that are involved in this obligate, stereotyped, pathway of capsid assembly. Additionally, the cell-free system offers the advantage that it allows “deconstruction” of any virus by determination of which host proteins the virus utilizes for capsid assembly without regard to conditions necessary to propagate or grow the virus per se and by use of only the viral nucleic acid that encodes the viral protein(s) that are involved in capsid assembly, thereby eliminating exposure of laboratory personnel to infectious virus. In this system, which faithfully reproduces what happens within a cell only more slowly, the assembly intermediates can be detected and enriched. The invention has the advantage that host proteins and viral proteins involved in capsid assembly can be identified even before the ability to culture the virus has been established, and/or the virus has been identified, and it also can be used for viruses that lack cell culture systems that produce high titers of virus.

This method for cell-free assembly of viral capsids has the additional advantage that a library can be developed that correlates the identity of host factors, including such characteristics as their amino acid sequence and/or any antibodies that inhibit capsid assembly with the particular viruses or families of viruses that use these host factors and for naturally occurring viruses, this information can be further cross-referenced with the identify of the virus and/or virus family. The host factor characteristics can additionally be cross-referenced to information relating to small molecules that inhibit capsid assembly for a virus that uses the particular host protein, so that by identifying the host protein, a treatment modality also can be identified. An additional advantage of this system is that even if a virus has been genetically altered and/or has mutated and/or is a synthetic virus, because it must still interact with host protein(s) in order to produce capsids, the viral protein binding site for a host protein required for capsid assembly will have been conserved and by identification of the host protein in an assembly intermediate, a treatment modality can be determined based upon that identification. This can be particularly useful when antibody epitopes have been altered and the virus is no longer recognized by existing antibodies, or where no antibodies to a particular virus exist.

Both host proteins and assembly intermediates are candidate antiviral targets. Thus another advantage of the subject invention is that the assembly intermediates can be isolated and used in the design of drugs (including peptides and antibodies) and vaccines that interfere with progression from one intermediate to the next, in the design of drugs that act by inhibiting host cell machinery involved in capsid formation, and in the design of assay systems that examine the efficacy and mechanism of action of drugs that inhibit capsid formation even in the absence of knowledge concerning the identity of the virus itself. Additionally, if the target for the antiviral drug is a host protein rather than a viral protein, there is a decreased likelihood of the development of viral resistance to such a drug. Another advantage of the subject invention is that pieces of genomic nucleic acid can be encapsidated into the capsids produced in the cell-free system by adding such nucleic acid to the system. This feature of the invention can be used to design drugs that interfere with encapsidation and in the design of assay systems that examine the mechanism of actions of drugs that inhibit encapsidation.

To produce viral capsid assembly intermediates, a cell-free translation system is used. Known in the art are a number of in vitro translation systems, the basic requirements of which have been well studied (Erickson and Blobel, Methods Enzymol (1983) 96:38-50; Merrick, W. C., Methods Enzymol. (1983) 101:606-615; Spirin et al. Science (1988) 242:1162-1164). Examples include wheat germ extract and rabbit reticulocyte extract, available from commercial suppliers such as Promega (Madison, Wis.), as well as high speed supernatants formed from such extracts. While the cell-free translation mixture can be derived from any of a number of cell types known in the art that contain the necessary components for capsid assembly, the present invention is exemplified using wheat germ cell-free extract which is prepared from the germ of wheat of different strains. (Erickson and Blobel (1983) Methods Enzymol 96, 38-50.). For example, necessary components of the cell-free extract for HIV capsid formation include a protein that binds to a 23c antibody; rabbit reticulocyte extract does not support production of HIV capsids in the absence of added host factor 68 (HP68.). Therefore, depending upon the virus involved, in some instances it may be necessary to supplement the cell-free system with exogenous proteins, such as host proteins, which facilitate the assembly of capsid intermediates. The need for addition of exogenous proteins for a particular virus can be determined empirically. The extract is the source of factors known to be required for translation, plus factors that have not yet been defined and may be required for assembly. While these extracts contain a mixture of membrane vesicles derived from plasma membrane and endoplasmic reticulum (ER) to which proteins can be targeted, ER vesicles that are capable of translocation are generally not present in significant quantities in the extract and are typically supplemented by adding exogenous membranes, such as dog pancreas membranes. As shown in this application, these capsid assembly systems closely reproduce capsid events that occur in vivo (also see Molla et al., (1991) Science 254, 1647-51, and Molla et al., (1993) Dev Biol Stand 78, 39-53.)

The components of cell-free assembly systems that have been used for making HCV, HBV, HIV-1, M-PMV, and other capsids have similarities and differences that reflect differences in virion morphogenesis. As an example, some viruses, such as HIV, have myristolated intermediaries, therefore it is necessary to add sufficient myristoyl coenzyme A (MCoA) to the system to enable assembly of capsids should the unknown virus be one that requires this component. The amount of myristoyl coenzyme A that is used to supplement the cell free translation mixture is that- which is sufficient to support capsid formation. While the concentration required varies according to the particular experimental conditions, in experiments carried out in support of the present invention, it was found that a concentration of between about 0.1 and 100 μM, and preferably between about 5 and 30 μM, supports HIV capsid formation.

Some viruses require membrane proteins for capsid assembly and appropriate membranes can be added to the cell-free translation mixture, including detergent-sensitive, detergent-insensitive, and host protein fractions described below, or it may be supplemented with such fractions. As an example, for HIV when membranes present in the cell free translation mixture are solubilized by addition of detergent, assembly of the HIV capsid is sensitive to addition of detergent above but not below the critical micelle concentration. This observation is consistent with a role for membranes being required at a particular step in capsid assembly. Furthermore, HIV capsid assembly is improved by the presence of a cellular component that has a sedimentation value greater than 90 S in a sucrose gradient and is insensitive to extraction with at least 0.5% “NIKKOL”. The term “detergent-sensitive fraction” refers to a component most likely containing a membrane lipid bilayer that is present in a standard wheat germ extract prepared according to the methods described by Erickson and Blobel (1983) (Methods in Enzymalogy Val 96), which component is deactivated with reference to supporting HIV capsid assembly when a concentration of 0.1% (wt/vol) “NIKKOL” is added to the extract. It is appreciated that such a detergent-sensitive factor can be present in extracts of other cells similarly prepared, or can be prepared independently from a separate cell extract, and then added to a cell-free translation system.

While both myristoylation machinery and membranes must be present in the cell-free system for viruses such as HIV, but are not required for capsid assembly for viruses such as HBV and HCV because the structural proteins are not myristoylated and targeting to the membrane is thought to occur after capsid assembly, the presence of these components in the cell-free translation system does not negatively affect viral capsid assembly for viruses that do not require them and these components therefore can be included in the cell-free system for making capsids of unknown viruses.

Methods known in the art are used to maintain energy levels in the cell-free system sufficient to maintain protein synthesis, for example, by adding additional nucleotide energy sources during the reaction or by addition of an energy source, such as creatine phosphate/creatine phosphokinase. The ATP and GTP concentrations present in the standard translation mixture, generally between about 0.1 and 10 mM, more preferably between about 0.5 and 2 mM, are sufficient to support both protein synthesis and capsid formation, which may require additional energy input. Generally, the reaction mixture prepared in accordance with the present invention can be titered with a sufficient amount of ATP and/or GTP to support production of a concentration of about 10 picomolar viral protein in the system.

Assembly of immature capsids in the cell-free system requires expression of only the particular viral protein(s) that are involved in capsid assembly. A sample containing an unknown virus or a bodily fluid of an individual infected with an unknown virus, or infected cells from the individual, is used as a source of viral nucleic acid encoding the capsid protein(s) for the virus. The fluid may be any bodily fluid including blood, serum, plasma, lymphatic fluid, urine, sputum, cerebrospinal fluid, or a purulent specimen. The genomes for many known viruses such as Ebola, smallpox, and Venezuela encephalitis virus, have been sequenced, for example see http://www.ncbi.nim.nih.gov:80/entrez/query.fcgi?db=Genome.<http://www.ncbi.nim.nih. gov:80/entrez/query.fcgi?db=Genome>. For unknown viruses or for those for which the genome has not been sequenced, the viral genome is cloned and sequenced and the capsid gene identified by sequence homology to known viral capsid genes.

Nucleic acids encoding viral proteins involved in capsid assembly can be obtained by amplification using the polymerase chain reaction (PCR). Primer sets that encompass the necessary nucleic acid sequences are designed based on sequence data for nucleic acid encoding capsid proteins of all known viruses, both sense and antisense strands. Since the coding sequences for unknown viruses may not be identical to known sequences but are likely to be related to sequences known to encode viral proteins involved in capsid assembly, consensus-degenerate hybrid oligonucleotide primers are used (see for example Rose et al., Nucleic Acids Research (1998) 26:1628-1635, which disclosure is incorporated herein by reference).

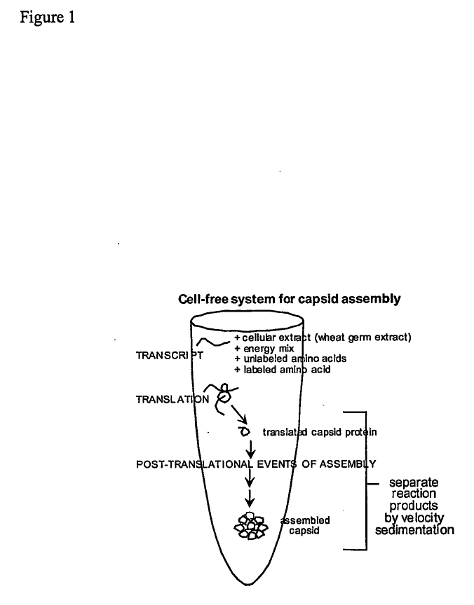

In the capsid assembly system (see FIG. 1), the cell-free translation mixture is programmed with capsid transcript for the unknown virus that is synthesized in vitro. The term “programmed with” means addition of mRNA that encodes viral capsid proteins to the cell-free translation mixture. Suitable mRNA preparations include a capped RNA transcript produced in vitro using the mMESSAGE mMACHINE kit (Albion). RNA molecules also can be generated in the same reaction vessel as is used for the translation reaction by addition of SP6 or T7 polymerase to the reaction mixture, along with the viral capsid protein coding region or cDNA.

After incubation for a time sufficient to produce capsids, products of the cell-free reaction are analyzed to determine sedimentation (S) value (which assesses size and shape of the particle), buoyant density (which indicates the density of the particle) and electron microscopy appearance. Together these form a sensitive set of measurements for integrity of capsid formation. A fourth criterion (resistance to protease digestion) also can be used. To confirm that the de-enveloped particles obtained represent the desired viral capsids, the fractions containing de-enveloped capsids from the velocity sedimentation gradient are analyzed by equilibrium centrifugation on CsC1 and the buoyant density compared with that of capsids (without envelopes) produced in infected cells if such are available. Production of capsids is confirmation that the viral nucleic acid identified encodes a capsid protein.

The cell free capsid assembly reaction described above can be extended to include packaging of nucleic acid, by addition of genomic nucleic acid or fragments thereof during the capsid assembly reaction. Addition and monitoring of encapsidation provides an additional parameter of particle formation that can be exploited in drug screening assays, in accordance with the present invention. The nucleic acid preferably is greater than about 1,000 nucleotides in length and is subcloned into a transcription vector. A corresponding RNA molecule is then produced by standard in vivo transcription procedures. This is added to the reaction mixture described above, at the beginning of the incubation period. Although the final concentration of RNA molecule present in the mixture will vary, the volume in which such molecule is added to the reaction mixture should be less than about 10% of the total volume.

Capsid assembly intermediates can be formed in a number of ways, such as by blocking the production of capsids in the cell-free assembly system by adding specific assembly blockers (e.g. apyrase to block ATP) or by subtraction of a key component, such as Myristoyl coA (for a virus which requires it) from the reaction. In this way, one or more assembly intermediates is produced in large quantity. The assembly intermediates then are analysed to determine the components of the complex, which generally include at least one host cell-derived assembly protein or chaperone. The presence of such a protein is detected by any of a number of means, for example by immunoprecipitation of the host protein-assembly intermediate complex using antibodies that bind to known host cell chaperones.

The host protein is separated from the assembly intermediate complex, for example by denaturation and the biochemical characteristics of the host cell protein are determined. The biochemical characteristics that are profiled include identifying immunoreactivity with monoclonal antibody(s) to known viral chaperones for example by screening phage display libraries and sequencing of the protein. The sequence is evaluated to determine whether it contains amino acid sequences from known viral chaperones and whether there are any homologues to the host protein, including wheat germ and primate homologues, particularly human. Human homologues can be identified using degenerate primers to the identified sequence, or other chaperone proteins identified in a cell free system that bind to the capsid assembly intermediates, and then cloned into an expression vector. Translation products from these expression vectors are tested in a cell free system to determine their ability to bind capsid assembly proteins by immunopurification. The protein is further characterized by molecular weight for example, as assessed by SDS-PAGE.

If monoclonal antibodies to the host proteins are not available, they are prepared by any number of methods which are known to those skilled in the art and previously described (see, for example, Kohler et al., Nature, 256: 495497 (1975) and Eur. J. Immunol. 6:511-519 (1976); Milstein et al., Nature 266: 550-552 (1977), Koprowski et al., U.S. Pat. No. 4,172,124; Harlow, E. and D. Lane, 1988, Antibodies: A Laboratory Manual, (Cold Spring Harbor Laboratory Press, Cold Spring Harbor, N.Y. (1989); Current Protocols In Moleclular Biology, Vol. 2 (Supplement 27, Summer '94), Ausubel, F. M. et al., Eds., (John Wiley & Sons: New York, N.Y.), Chapter 11, (1991)). Generally, a hybridoma is produced by fusing a suitable immortal cell line (e.g., a myeloma cell line) with antibody-producing cells (for example, lymphocytes derived from the spleen or lymph nodes of an animal immunized with an antigen of interest). The cells resulting from a fusion of immune cells and lymphoma cells, generally referred to as hybridomas, can be isolated using selective culture conditions, and then cloned by limiting dilution. Cells which produce antibodies with the desired binding properties are selected by a suitable assay, such as a serological assay, including enzyme-linked immunosorbent assay (ELISA).

Functional binding fragments of monoclonal antibodies also can-be-produced by, for example, enzymatic cleavage or by recombinant techniques. Enzymatic cleavage methods include papain or pepsin cleavage to generate Fab or F(ab′)₂ fragments, respectively. Antibodies also can be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain. Functional fragments of the monoclonal antibodies retain at least one binding function and/or modulation function of the full-length antibody from which they are derived. Preferred functional fragments retain an antigen-binding function of a corresponding full-length antibody (e.g., retain the ability to bind an epitope of a host protein). In another embodiment, functional fragments retain the ability to inhibit one or more functions characteristic of the host protein, such as a binding activity.

Antibodies also can be produced using knock-out mice that lack a functional gene for the host protein. Knockout mice can be produced using standard techniques known to those skilled in the art (Capecchi, Science (1989) 244:1288; Koller et al. Annu Rev Immunol (1992) 10:705-30; Deng et al. Arch Neurol (2000) 57:1695-1702). A targeting vector is constructed which, in addition to containing a fragment of the gene to be knocked out, generally contains an antibiotic resistance gene, preferably neomycin, to select for homologous recombination and a viral thymidine kinase (TK) gene. Alternatively, the gene encoding diphtheria toxin (DTA) can be used to select against random insertion. The vector is designed so that if homologous recombination occurs the neomycin resistance gene is integrated into the genome, but the TK or DTA gene is always lost. Murine embryonic stem (ES) cells are transfected with the linearized targeting vector and through homologous recombination recombine at the locus of the targeted gene to be knocked out. Murine ES cells are grown in the presence of neomycin and ganciclovir (for TK), a drug that is metabolized by TK to produce a lethal product. Thus cells that have undergone homologous recombination are resistant to both neomycin and ganciclovir. Vectors containing DTA kill any cell that codes for the gene, so no additional drug is required in the cell culture medium. Southern blotting hybridization and PCR are used to verify the homologous recombination event, techniques well known to those skilled in the art.

To generate a mouse carrying a disrupted targeted gene, positive ES cells are propagated in culture to differentiate and the resulting blastocyte is implanted into a pseudopregnant female. Alternatively the ES cells-are injected back into the blastocoelic cavity of a preimplantation mouse embryo and the blastocyte is then surgically implanted. The transfected ES cells and recipient blastocytes can be from mice with different coat colors, so that chimeric offspring can be easily identified. Through breeding techniques homozygous knockout mice are generated. Tissue from these mice is tested to verify the homozygous knockout for the targeted gene, for example using PCR and Southern blotting hybridization.

In an alternate method, gene targeting using antisense technology can be used (Bergot et al., JBC (2000) 275:17605-17610). The homozygous knockout mice are immunized with purified host protein peptides, both native and denatured recombinant protein. Following subsequent boosts, at 3 and 6 weeks, with the immunogen, the mice are sacrificed and spleens taken and fusion to myeloma cells carried out (Korth et al. Methods in Enzymol. (1999) 309:106). Antibodies from individual hybridomas are screened for conformational specificity, i.e., binding with substantial specificity to a single conformer. The screening process is carried out with radiolabeled protein products produced in the cell-free translation system or radiolabeled media or cell extracts chosen to enrich one versus another conformer. These products are immunoprecipitated using hybridoma supernatant and run on a SDS-PAGE gel. Preferably cell-free extracts are used due to the possibility that the use of transfected cells would result in protein-protein interactions which would block antibodies from binding a specific epitope, thus masking a potential conformer. The use of an immunoprecipitation screen with radiolabled translation products, the conformation of which has been skewed (e.g. by viral infection), is the key that distinguishes this screen from a conventional approach to monoclonal antibody production. The use of 96 well plates for screening streamlines the process, allowing a single technician to screen up to many hundreds of individual hybridomas in a single day). The procedures above also can be used to prepare antibodies to capsid proteins, and to assembly intermediates.

Of particular interest are antibodies to a binding site on the host protein for a viral capsid protein and/or on a viral capsid protein for a host protein. Known antibodies to host proteins include antibodies to the t complex polypeptide 1 (TCP-1) (see Willison et al, (1989) Cell, 57: 621-632. Several antibodies have been prepared that recognize different epitopes on TCP-1 and that also recognize epitopes on HIV-1, HCV, HBV and N-MPV (see Example 19). Antibodies to the matrix assembly (MA) domain of M-PMV have been reported to inhibit capsid assembly and therefore may bind to either a host protein and/or a viral capsid protein involved in capsid assembly. Binding between capsid proteins and host proteins in capsid assembly intermediates can be analysed and the binding sites identified using technology developed by Biacore AB (www.biacore.com).

The cell-free system can be used to identify possible compounds that inhibit formation of capsid assembly intermediates necessary for the production of viral capsids, which can then be screened for their ability to inhibit viral replication. Upon identification of compounds of interest, the compounds are tested in human cells under similar conditions. The assay can be set up according to any of a number of formats. Two different types of assays can be used either alone or in combination. To screen for compounds that block or impair viral capsid formation, monoclonal or polyclonal antibodies are used directly. High throughput screening of compounds for lead candidates can be carried out using any of a variety of techniques known to those of skill is the act such as, for example by screening for inhibition and/or reversal of the distinctive immunofluorescent pattern of binding that is observed between viral capsid proteins and host proteins. These lead compounds are then further tested for specificity. In another such assay, cell-free translation and assembly is carried out in the presence or absence of a candidate drug in a liquid phase. The reaction product is then added to a solid phase immunocapture site coated with antibodies specific for one or more of the viral capsid assembly intermediates originally identified using the cell-free translation system, or the complete viral capsid. In this way, the precise point of assembly interference of the drug can be determined. Lead compounds can first be identified based on searches of databases for compounds likely to bind an active site involved in capsid assembly then tested in a cell-free system for inihibition of capsid formation. Such information can be used to identify potential treatments, or combination therapeutics against viral infection, by targeting different aspects of viral replication.

A compound that is found to block viral capsid formation by binding to an active site on an assembly intermediate and/or host protein in the cell-free system is then tested in mammalian cells infected with the unknown virus. Preferably, compounds also are screened for toxicity, including host stress responses such as activation of heat shock proteins (HSP) 70, 80, 90, 94 and caspases (Flores et al., J. Nueroscience (2000) 20:7622-30). Methods for evaluating activation of these proteins are well known to those skilled in the art.

The cell-free translation/assembly system can be used to produce large quantities of wild-type viral capsids, capsid intermediates or mutant capsids which can be used, for example to produce vaccines. The system also can be used as a means of identifying compounds that inhibit capsid formation, by adding to a cell a compound that has been selected for its ability to inhibit capsid formation or formation of capsid intermediate(s) in the cell-free translation system. For enveloped viruses, the cell free system can be used with plasmids that code for the entire viral genome, except for envelope protein. Thus, the invention includes a method of encapsidating genomic viral nucleic or fragments thereof. Genomic nucleic acid or a fragment or a plasmid encoding viral nucleic acid is added to such a system, and is encapsidated during the reaction process. Antibodies that are produced find utility as reagents in screening assays that assess the status of viral capsid formation or in assays used for screening for drugs that interfere with viral capsid formation, and also can be used as a diagnostic for determining the identity of a virus causing a viral infection. Genes encoding the variable region of antibodies to the viral capsid proteins can be inserted into an appropriate vector for transducing cells that are the target of the unknown virus, and the cells transduced to express the intrabody to the viral capsid protein. See for example Goncalves et al (2002) J. Biol. Chem. 35: 32036-32045 which describes the functional neutralization of HIV-1 Vif protein by intracellular immunization and consequent inhibition of viral replication.

By detecting and characterizing host proteins and/or assembly intermediates associated with a number of viruses or families of viruses, a library of the various host proteins and/or assembly intermediates can be developed in which the members of the library are individual viruses or families of viruses. Each member is cross-referenced with the biochemical characteristics of the host proteins and/or assembly intermediates for that virus or family of viruses. The characteristics include, for example, the amino acid sequence of the host protein(s), antibodies that bind to the host protein(s) and/or assembly intermediates and preferably inhibit capsid assembly, the nucleic acid sequence of the viral capsid genes, PCR primer sets useful for amplifying these genes, the physicochemical characteristics of the viral capsids produced using the cell-free translation system, such as the sedimentation coefficient, buoyant density and appearance using electron microscopy, and any small molecules that inhibit capsid assembly. The library can be used to determine a definitive disease diagnosis when there is at least a substantial similarity between the characteristics of the unknown virus and a member of the library.

A treatment protocol for an individual infected with an unknown virus can be identified for those infected, even if the identity of the virus is unknown or the only characteristic in common between the unknown virus and a member of the library is the host protein or a portion thereof involved in binding to viral protein(s) during capsid assembly. As an example, inhibition of production of capsids of the unknown virus in the cell free system with a test compund is an indication that this test compound can be used as a treatment against the virus. The host protein and/or assembly intermediates that are identified using the cell-free system can be screened using a panel that includes antibodies or functional fragments thereof to the members of the library of host proteins and/or assembly intermediates associated with capsid assembly in other viruses. Preferably, the panel is immobilized on a solid support. Generally the antibody is a monoclonal antibody or fragment thereof specific for the host protein or assembly intermediate. The monoclonal antibody or binding fragment is labeled with a detectable label, for example, a radiolabel or an enzyme label. Examples of enzyme labels that can be linked to the antibody include horseradish peroxidase, alkaline phosphatase, and urease, and methods for linking enzymes with antibodies are well known in the art. The label may be detected using methods well known to those skilled in the art, such as radiography, or serological methods including ELISA or blotting methods. The presence of the label is indicative of the presence of at least one protein or assembly intermediate involved in capsid assembly that shares an epitope with a member of the library. If the biochemical characteristics for the member include information as to means for inhibiting capsid assembly by interfering with binding between the host protein and viral protein(s) involved in capsid assembly, such a means will be efficacious in inhibiting capsid assembly of the unknown virus.

The following examples illustrate, but in no way are intended to limit, the present invention.

### EXAMPLES

### Materials

1. Chemicals

Chemical sources are as follows, unless otherwise indicated below: Nonidet P40 (NP40) was obtained from Sigma Chemical Co. (St. Louis, Mo.). “NIKKOL” was obtained from Nikko Chemicals Ltd. (Tokyo, Japan). Wheat Germ was obtained from General Mills (Vallejo, Calif.). Myristoyl Coenzyme A (MCoA) was obtained from Sigma Chemical Co. (St. Louis, Mo.).

2. Plasmid Constructions

All plasmid constructions for cell-free transcription were made using polymerase chain reactions (PCR) and other standard nucleic acid techniques (Sambrook, J., et al., in Molecular Cloning. A Laboratory Manual). Plasmid vectors were derived from SP64 (Promega) into which the 5′ untranslated region of Xenopus globin had been inserted at the Hind Ill site (Melton, D.A., et al., Nucleic Acids Res. 12:7035-7056 (1984)). The gag open reading frame (ORF) from HIV genomic DNA (a kind gift of Jay Levy; University of California, San Francisco) was introduced downstream from the SP6 promoter and the globin untranslated region. The GΔA mutation was made by changing glycine at position 2 of Gag to alanine using PCR (Gottlinger, H. G., et al., Proc. Natl. Acad. Sci. 86:5781-5785 (1989)). The Pr46 mutant was made by introducing a stop codon after gly 435 (removes p6); Pr4l has a stop codon after arg 361 (in the C terminal region of p24). These truncation mutants are comparable to those described by Jowett, J. B. M., et al., J Gen. Virol. 73:3079-3086 (1992), incorporated herein by reference. To make the D2 mutant amino acids from gly 250 to val 260 were deleted (as in Hockley, D. J. et al., J. Gen. Virol. 75:2985-2997 (1994); Zhao, Y., et al., Virology 199:403-408 (1994)). All changes engineered by PCR were verified by DNA sequencing. The plasmid, pBRUΔenv, which encodes for the entire HIV-1 genome except a deletion in envelope, was made and used as previously described (Kimpton et al. J. Virology (1992) 66:2232-9). The plasmid, WGHP68-Tr1, encodes a 379 amino acid truncated form of HP68 with a stop codon before the second nucleotide-binding domain (Arrow, FIG. 6). This plasmid encodes the N-terminal two-thirds of WGHP68 and produces the expected 43 kD protein when transfected into cells (FIG. 7)

3. ³⁵—S Energy Mix

³⁵—S Energy Mix (5× stock) contains 5 mM ATP (Boehringer Mannheim), 5 mM GTP (Boehringer Mannheim), 60 mM Creatine Phosphate (Boehringer Mannheim), 19 amino acid mix minus methionine (each amino acid except methionine; each is at 0.2 mM),;³⁵—S methionine 1 mCurie (ICN) in a volume of-200 microliters at a pH of 7.6 with 2 M Tris base.

4. Compensating Buffer

The Compensating Buffer (10×) contains 40 mM HEPES-KOH, at a pH of 7.6 (U.S. Biochemicals), 1.2 M KAcetate (Sigma Chemical Co.), and 2 mM EDTA (Mallinckrodt Chemicals, Paris, Ky.).

### Example 1

### Cell Free Protein Synthesis

1. In vitro Transcription

The plasmid containing the Gag coding region was linearized at the EcoRI site (as described in the NEB catalogue). The linearized plasmid was purified by phenol-chloroform extraction (as described in Sambrook, J., et al., in Molecular Cloning. A Laboratory Manual) and this plasmid was adjusted to a DNA concentration of 2.0 mg/ml. Transcription was carried out using a reaction that contained: 40 mM Tris Ac (7.5), 6 mM Mg Ac, 2 mM Spermidine, 0.5 mM ATP, 0.5 mM CTP, 0.5 mM UTP, 0.1 mM GTP, 0.5 mM diguanosine triphosphate (cap), 10 mM Dithiothreitol, 0.2 mg/ml transfer RNA (Sigma Chemical Co.), 0.8 units/microliter RNAse inhibitor (Promega), 0.4 units per p1 of SP6 Polymerase (NEB). Mutant DNAs were prepared as described by Gottlinger, H. G., et al., Proc. Natl. Acad. Sci. 86:5781-5785 (1989); Jowett, J. B. M., et al., J. Gen. Virol. 73:3079-3086 (1992); Hockley, D. J. et al., J. Gen. Virol. 75:2985-2997 (1994); or Zhao, Y., et al., Virology 199:403-408 (1994); these publications are incorporated herein by reference.

2. Cell-Free Translation System

Translation of the transcription products was carried out in wheat germ extract containing ³⁵S methionine (ICN Pharmaceuticals, Costa Mesa, Calif.). Wheat germ was obtained from General Mills. Wheat germ extract was prepared as described by Erickson and Blobel (1983) supra with indicated modifications. Three grams of wheat germ were placed in a mortar and ground in 10 ml homogenization buffer (100 mM K-acetate, 1 mM Mg-acetate, 2 mM CaC1₂, 40 mM HEPES buffer, pH 7.5 (Sigma Chemicals, St. Louis, Mo.), 4 mM dithiothreitol) to a thick paste. The homogenate was scraped into a chilled centrifuge tube and centrifuged at 4° C. for 10 min at 23,000×g. The resulting supernatant was centrifuged again under these conditions to provide an S23 wheat germ extract. Improved assembly was obtained when the S23 wheat germ extract was further subjected to ultracentrifugation at 50,000 rpm in the TLA 100 rotor (100,000×g) (Beckman Instruments, Palo Alto, Calif.) for 15 min at 4° C. and the supernatant used for in vitro translation. This improvement provided 2-3× the yield obtained in comparable reactions using the S23 wheat germ extract. This supernatant is referred to herein as a “high speed wheat germ extract supernatant”. Reactions were performed as previously described (Lingappa, J. R., et al., J. Cell. Biol. (1984) 125:99-111), except for modifications noted below.

A 25 μl wheat germ transcription/translation reaction mixture contained: 5 μl Gag transcript, 5 μl ³⁵—S Energy Mix 5× stock (Sigma Chemical Co., St. Louis, Mo.), 2.5 μl Compensating Buffer (Sigma Chemical Co.), 1.0 μl 40 mM MgAcetate (Sigma Chemical Co.), 2.0 μl 125 μM Myristoyl CoA (made up in 20 mM Tris Acetate, pH 7.6; Sigma Chemical Co.), 3.75 μl 20 mM Tris Acetate buffer, p11 7.6 (U.S. Biochemicals; Cleveland, Ohio), 0.25 μl creatine kinase (4 mg/ml stock in 50% glycerol, 10 mM Tris Acetate; Boehringer Mannheim, Indianapolis, Ind.), 0.25 μl bovine tRNA (10 mg/ml stock; Sigma Chemical Co.), and 0.25 μl RNAse Inhibitor (20 units/50; Promega).

Myristoyl coenzyme A (MCoA; Sigma, St. Louis, Mo.) was added at a concentration of 10 μM at the start of translation when indicated. Translation reactions ranged in volume from 20 to 100 μl and were incubated at 25° C. for 150 min. Some reactions were adjusted to a final concentration of the following agents at times indicated in the Figures and specification: 0.2 μM emetine (Sigma); 1.0 units apyrase (Sigma) per mL translation; 0.002%, 0.1%, or 1.0% “NIKKOL”. Inpulse-chase experiments, translation reactions contained ³⁵S cysteine (Amersham Life Sciences, Cleveland, Ohio) for radiolabeling. After 4 min translation reaction time, 3 mM unlabeled cysteine was added, and the reaction was continued at 25° C. for variable chase times as indicated in the experiments described below. Protein synthesis was initiated in the cell-free translation/assembly system by adding an mRNA that encodes Gag Pr55 protein. Alternatively, when the system includes transcription means, such as SP6 or T7 polymerase, the reaction may be initiated by addition of DNA encoding the protein. Complete synthesis of protein and assembly into capsids is usually achieved within about 150 minutes.

3. Estimation of Sedimentation Coefficients

Estimates of S-values of Gag-containing complexes seen on 13 ml sucrose gradients were determined by the method of McEwen, C. R., Anal. Biochem. 20:114-149 (1967) using the following formula:
S=ΔI/ω²t
where S is the sedimentation coefficient of the particle in Svedberg units, ΔI is the time integral for sucrose at the separated zone minus the time integral for sucrose at the meniscus of the gradient, ω is rotor-speed in radians/sec. and t is time in sec. Values for I were determined for particles of a density of 1.3 g/cm3 and for a temperature of 5° C., according to tables published by McEwen, C. R., Anal. Biochem. 20:114-149 (1967). Calculated S values for different fractions in the gradients are labeled as markers above each gradient tracing shown herein. Markers such as BSA (5-S), macroglobulin (20-S), Hepatitis B Virus capsids (100-S), ribosomal subunits (40-S and 60-S), and polysomes (>100-S) were used to calibrate the gradients and to confirm the calculated S values. However, it should be noted that the S value assignments for each Gag-containing complex are approximate estimates and may vary by about ±10%.

### Example 2

### Translation of Gag Pr55 Protein in a Cell Free System

The purpose of this experiment was to show that capsids formed in the cell-free system described in Example 1 are substantially the same as those formed in cells. Cos-1 cells (University of California Cell Culture Facility) were transfected by the adenovirus—based method (Forsayeth, J. R. and Garcia, P. D., Biotechniques 17:354-358 (1994)), using plasmids pSVGagRRE-R (a mammalian expression vector that encodes Gag as well as the Rev response element required for expression of Gag in mammalian cells) and pSVRev (a mammalian expression vector that encodes the Rev gene, the product of which is required for expression of Gag in mammalian cells) (Smith, A. J., et al., J. Virol. 67:2266-2275 (1993)). These vectors were provided by D. Rekosh (University of Virginia). Cells were also transfected with pBRUΔenv. Four days after transfection, immature HIV particles were purified from the culture medium by sedimentation through a 4 ml 20% sucrose cushion in an SW 40 rotor at 29,000 rpm for 120 min (Mergener, K., et al., Virology 186:25-39 (1992)). The pellet was harvested, stored in aliquots at −80° C., and treated with 1% NP40 buffer just before use to remove envelopes. These de-enveloped authentic immature HIV capsids were used as standards and analyzed in parallel with the products of cell-free reactions by a variety of methods, including velocity sedimentation, equilibrium centrifugation, and electron microscopy.

Shown in FIG. 2 is a comparison of migration of the capsids through an isopycnic CsC1 gradient, where capsids formed in the cell-free translation/assembly system are shown in FIG. 2A, and capsids formed in transfected Cos cells are shown in FIG. 2B. Cell-free translation and assembly reactions containing 10 μM MCoA and ³⁵S methionine were programmed with HIV Gag transcript and incubated under the conditions detailed in Example 1. At the end of the reaction, samples were diluted into buffer containing 1% NP40 (a non-ionic detergent), and separated into. soluble and particulate fractions on sucrose step gradients, according to standard methods known in the art employing sucrose step or linear gradients as appropriate. The particulate fraction was collected and analyzed by velocity sedimentation on a 13-ml 15-60% linear sucrose gradient (Beckmnan SW40 Ti rotor, 35,000 rpm, 75-90 min). Fractions from the gradient were collected and subjected to sodium lauryl sulfate polyacrylamide gel electrophoresis (SDS-PAGE) analysis according to standard methods. Gag polypeptide present in the fractions was visualized by immunoblotting with a monoclonal antibody to Gag (Dako, Carpenteria, Calif.). Bound antibody was detected using an enhanced chemiluminescence system (Amersham). Band density was determined as described under image analysis below, and relative band densities were confirmed by quantitating films representing different exposure times.

A parallel analysis of the particulate fraction was performed by subjecting the particulate fraction to CsC1 gradient separation (2 ml isopycnic CsC1, 402.6 mg/ml; 50,000 rpm in a Beckman TLA 100 centrifuge) according to standard methods. Fractions were collected and assessed for Gag translation product (Pr55) (top of gradient is fraction 1, open circles, FIG. 2B). The fractions containing radiolabeled Pr55 were also subjected to SDS PAGE analysis; Gag content of the various fractions was estimated by scanning densitometry of autoradiographs made from the gels. Both conditions produced identical radiolabeled protein bands under these conditions. Material in the particulate fraction (>500-S) was further analyzed by a variety of methods as described below. Detergent-treated capsids-generated in the cell-free system and detergent-treated (de-enveloped) authentic capsids behaved as a relatively homogenous population of particles of approximately 750-S (compare FIGS. 2A and 2B), with a buoyant density of 1.36 g.cm-3. Additionally, cell-free-assembled capsids and the authentic standard were identical in size as judged by gel filtration. Electron microscopic analysis revealed that capsids made in the cell-free system were morphologically similar to authentic capsids released from transfected cells and had the expected diameter of approximately 100 nm (Gelderblom, H. R., AIDS 5:617-638 (1991)). Thus, radiolabeled Pr55 protein synthesized in the cell-free system assembles into particles that closely resemble authentic immature HIV capsids generated in transfected cells, as judged by EM appearance as well as the biochemical criteria of size, sedimentation coefficient, and buoyant density.

Translation of the HIV Gag transcript encoding Pr55 in the cell-free system resulted in the synthesis of approximately 2 ng Pr55 protein per microliter translation reaction. It is appreciated that increased production might be achieved, for example, by employing a continuous flow translation system (Spirin, A. S., et al., Science 242: 1162-1164 (1988)) augmented with the specific factors and components described above.

### Example 3

### Immunoprecipitation of Capsid Assembly Intermediates

Immunoprecipitation under native conditions was performed by diluting 2 μL samples of cell-free reactions into 30 μL of 1% NP40 buffer, and adding approximately 1.0 μg of one of monoclonal antibody 23 c (Institute for Cancer Research, London, UK; Stressgen, Vancouver, BC). Samples containing antibodies were incubated for one hour on ice, a 50% slurry of Protein G beads (Pierce, Rockford, Ill.) or Protein A Affigel (BioRad, Richmond, Calif.) was added, and incubations with constant mixing were performed for one hour at 4° C. Beads were washed twice in 1% NP 40 buffer containing 0.1 M Tris, pH 8.0, and then twice in wash buffer (0.1 M NaCl, 0.1 M Tris, pH 8.0, 4 mM MgAc). Proteins were eluted from the beads by boiling in 20 μL SDS sample buffer and were visualized by SDS-PAGE and autoradiography, according to methods well known in the art.

### Example 4

### Identification of HIV Capsid Intermediates

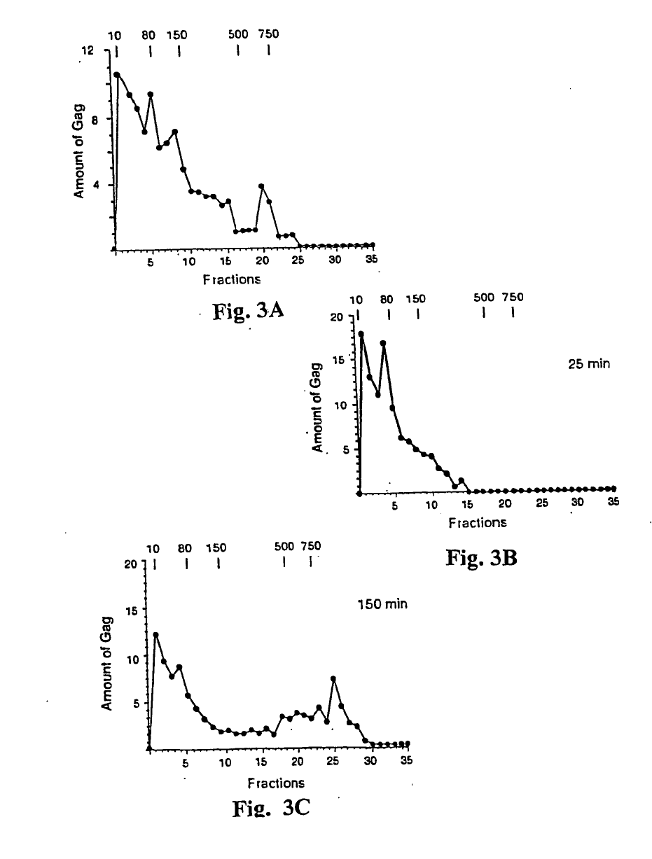

The purpose of this experiment was to use the cell-free system for detecting HIV assembly intermediates that would be otherwise difficult or impossible to detect. A continuously labeled cell-free reaction was analyzed by velocity sedimentation. Cell-free translation and assembly of Pr55 was performed as described in Example 1 above. Upon completion of the cell-free reaction, the products were diluted into 1% NP40 sample buffer on ice, and were analyzed by velocity sedimentation on 13 ml 15-60% sucrose gradients. Fractions were collected from the top of each gradient, and the amount of radiolabeled Pr55 protein in each fraction was determined and expressed as percent of total Pr55 protein present in the reaction. The calculated positions of 10S, 80S, 150S, 500S, and 750S complexes are indicated with markers above the figures (see FIG. 3A). 750S represents the position of authentic immature (de-enveloped) HIV capsids. The intermediate complexes having calculated sedimentation coefficients of 10S, 80S, 150S and 500S are referred to herein as intermediates A, B, C and D, respectively.

Further experiments indicated that the identified intermediates represent assembly intermediates, as evidenced by the observation that they were present in large quantities at early time points, and were diminished at later times during the reaction. Pulse-chase analysis was used to follow a small cohort of radiolabeled Pr55 chains over time during the assembly reaction. Cell-free translation and assembly of Pr55 was performed according to the methods set forth in Example 1, except that ³⁵S cysteine was used for radiolabeling. At 4 minutes into the translation reaction, an excess of unlabeled cysteine was added to the reaction so that no further radiolabeling would occur. Aliquots of the reaction were collected at 25 min (FIG. 3C) and 150 min (FIG. 3D) into the reaction. One microliter of each aliquot was analyzed by SDS-PAGE and AR to reveal the total amount of radiolabeled Pr55 translation product (indicated by arrow in FIG. 3B) present at each chase time. The remainder of the aliquots were diluted into 1% NP40 sample buffer on ice, and were analyzed by velocity sedimentation on 13 ml 15-60% sucrose gradients (FIGS. 3C and 3D respectively), in the manner described for FIG. 3A above. The total amount of radiolabeled Pr55 was the same at 25 min and 150 min into the pulse-chase reaction, indicating that neither further radiolabeling nor degradation of Pr55 chains occurred after 25 min, and confirming that the same population of Pr55 chains was being analyzed at both time points.

After 25 minutes of reaction time all of the radiolabeled Pr55 was found in complexes A, B, and C (FIG. 3C), with no radiolabeled Pr55 chains present in the region of completed 750S capsids. While complexes A and B appeared as peaks at approximately the 10S and 80S positions of the gradient, complex C appeared as a less distinct shoulder in approximately the 150S position. In marked contrast, examination of the assembly reaction at 150 minutes showed that a significant amount of radiolabeled Pr55 was assembled into completed capsids that migrated in the 750S position (FIG. 3D). Correspondingly, the amount of Pr55 in complexes A, B, and C was diminished by precisely the amount that was now found to be assembled, demonstrating that at least some of the material in complexes A, B, and C constituted intermediates in the biogenesis of completed 750S capsids.

At extremely short chase times (i.e., 13 min), when only some of the radiolabeled chains had completed synthesis, full length Pr55 chains were found exclusively in complex A on 13 ml sucrose gradients, while nascent chains that were not yet completed were in the form of polysomes of greater than 100S. Thus, polysome-associated nascent chains of Gag constituted the starting material in this pathway, and the 10S complex A, which contained completed Gag chains, was likely to be the first intermediate in the formation of immature capsids. Therefore, complexes B and C represent later assembly intermediates in the pathway of capsid formation.

As further confirmation that complexes A, B, and C constituted intermediates in HIV capsid assembly, blockade of assembly was studied to determine whether Gag chains accumulated in the form of complexes with S values corresponding to the S values of A, B and C, and whether blockade at different points along the pathway would result in accumulation of complexes A, B, and C in various combinations, as determined by the order of their appearance during the course of assembly. For example, if an ordered pathway of intermediates exists, then blockade at early points in the pathway should result in accumulation of one or two Gag-containing complexes corresponding to early putative assembly intermediates, while blockade at a very late point in the pathway would result in. accumulation of all of the putative assembly intermediates, but not the final completed capsid product.

Capsid assembly was disrupted by adding either apyrase post-translationally or detergent cotranslationally, and the reaction products were analyzed by velocity sedimentation. Material in fractions corresponding to the assembly intermediates and completed capsid were quantified and are presented in Table 1 below.

TABLE 1
Effect of Pharmacological Blockage on HIV Capsid Assembly

| | A | B/C | Final Capsid |
|---|---|---|---|
| Untreated | 2798 | 5046 | 739 |
| +apyrase | 2851 | 5999 | 133 |
| +detergent | 2656 | 6130 | 189 |

The untreated reaction contained Pr55 in complexes A, B, and C, as well as a peak in the final 750S capsid position, while the treated reactions contained no peak at the position of the final capsid product (Table 1). Treatment with either apyrase or detergent resulted in accumulation of additional material in complexes B and C, but did not result in accumulation of additional material in complex A. This is consistent with the idea that complexes B and C are the more immediate precursors of the 750S completed capsids, and that these interventions block the conversion of complexes B and C into the fully assembled capsid end-product.

### Example 5

### Host Cell Proteins involved in HIV Capsid Intermediate Formation

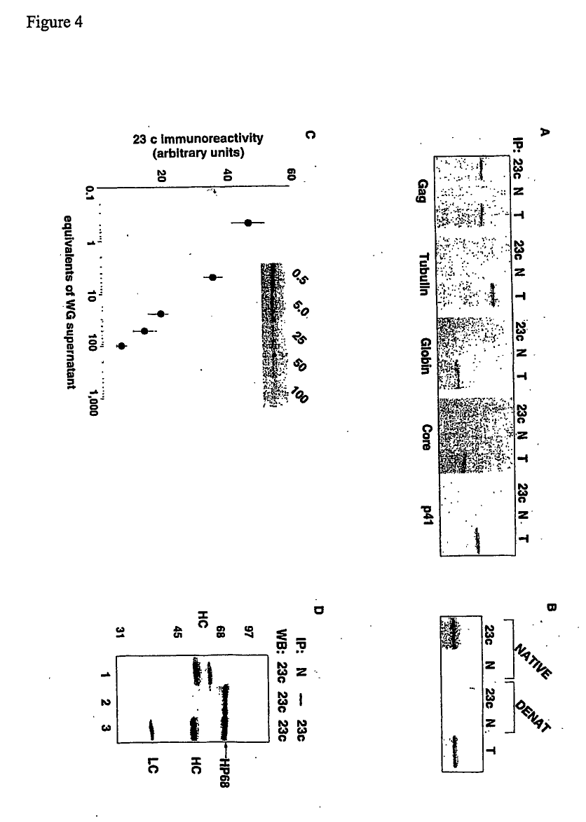

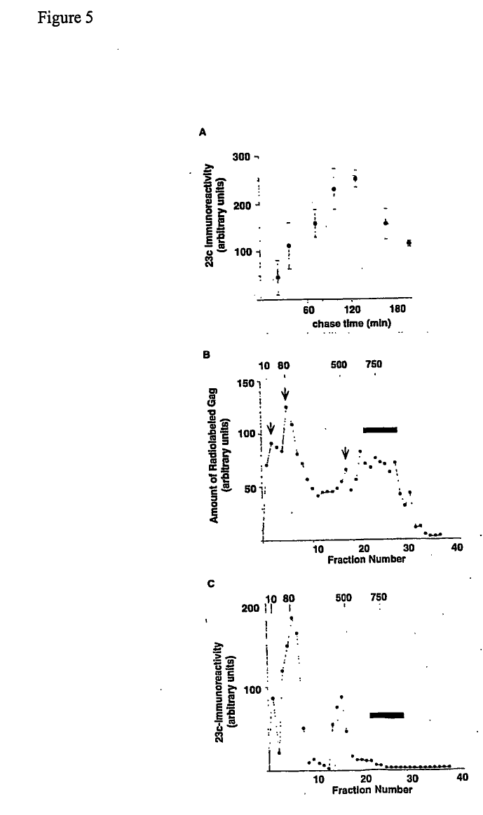

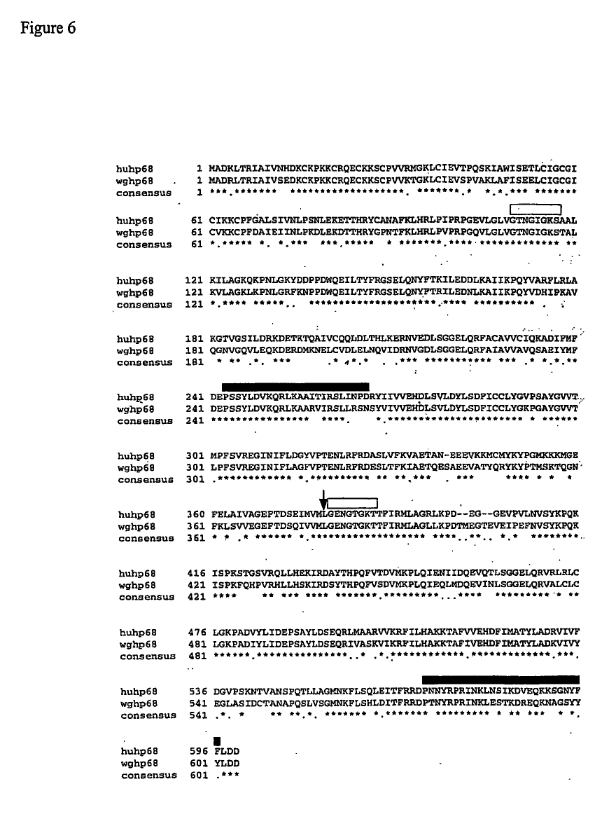

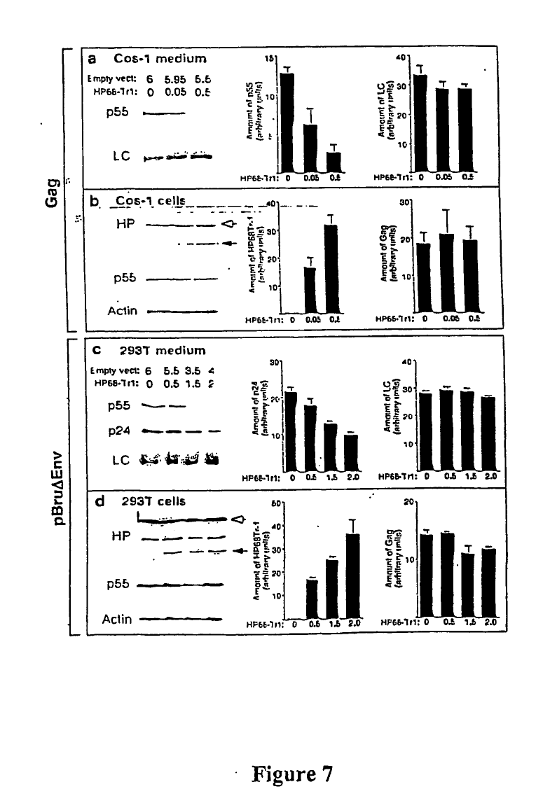

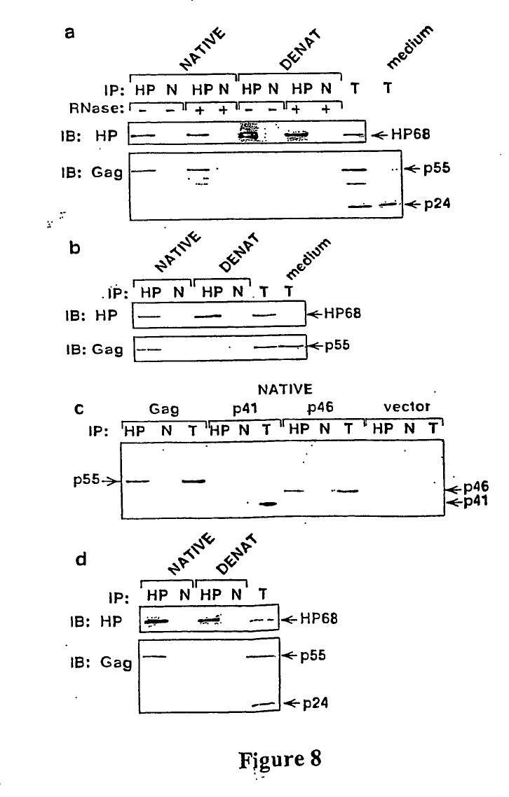

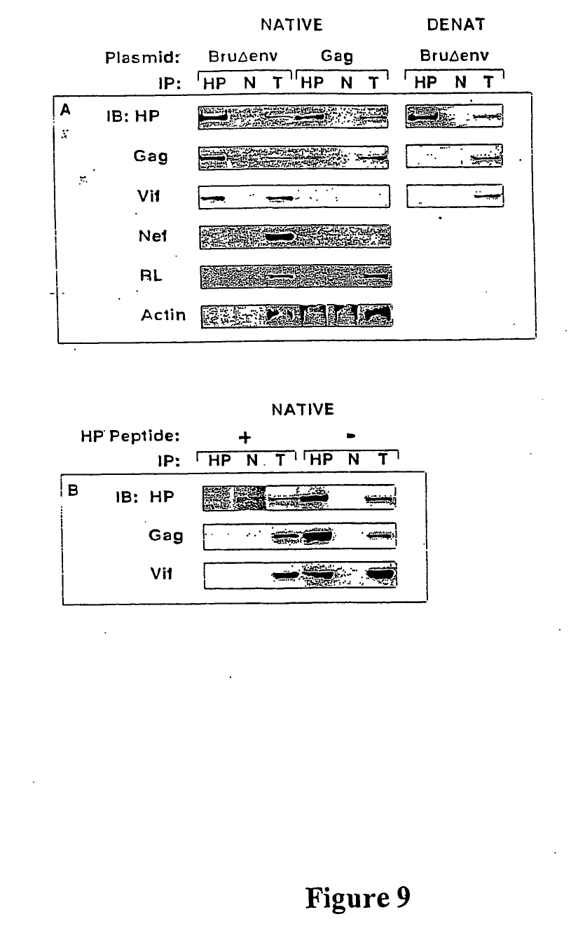

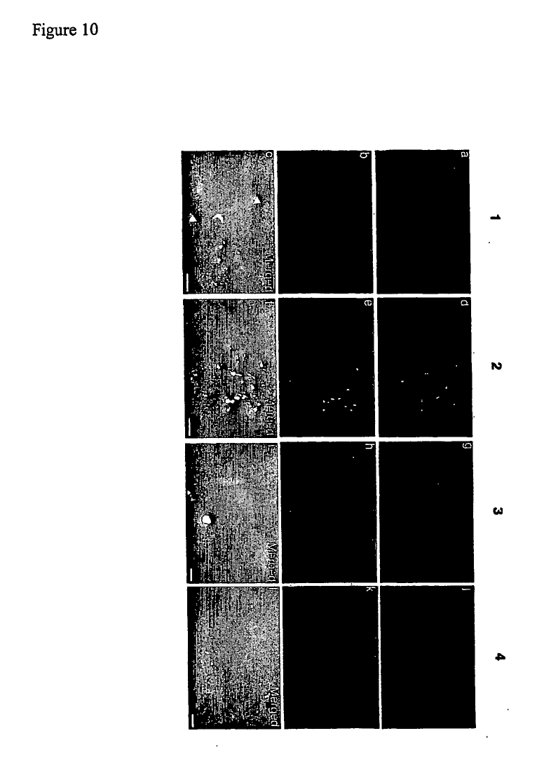

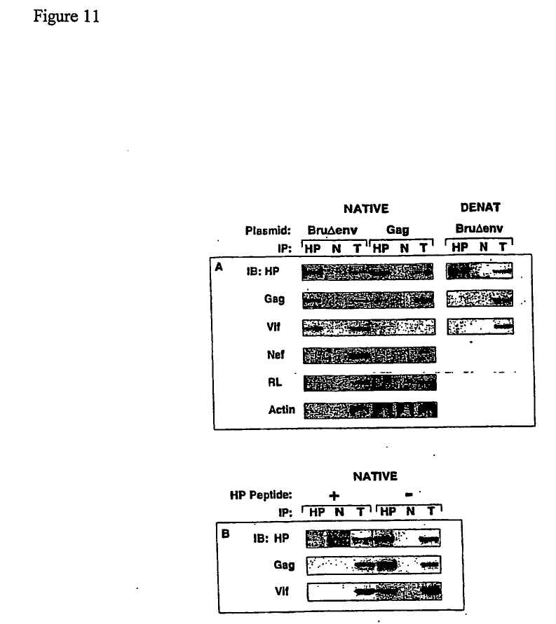

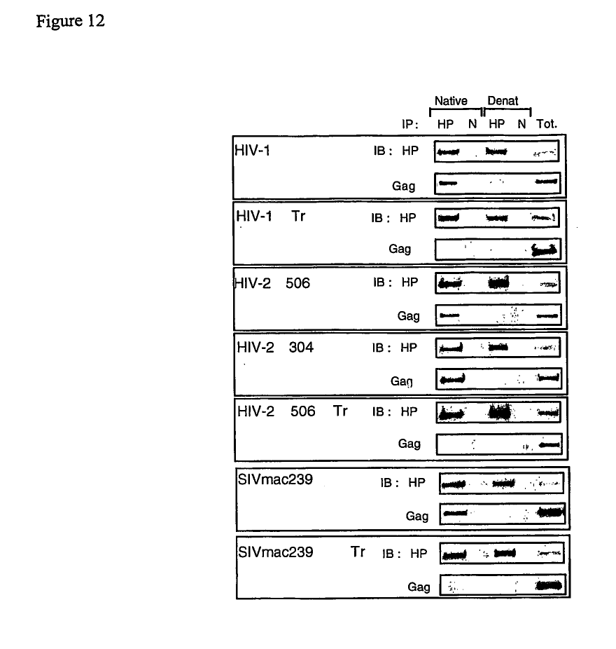

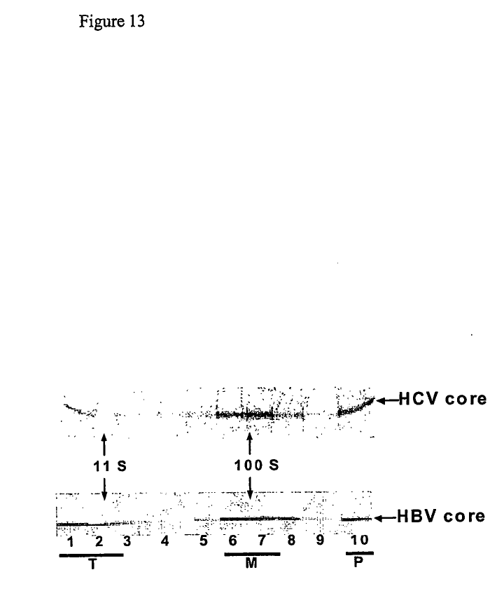

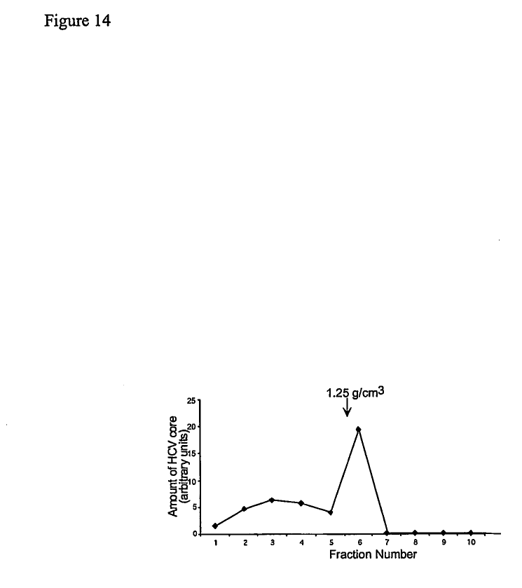

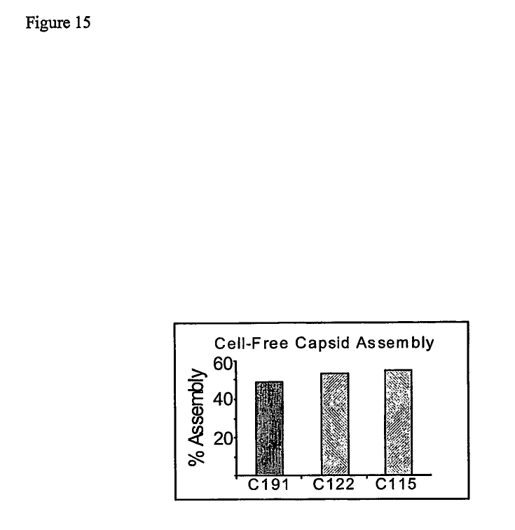

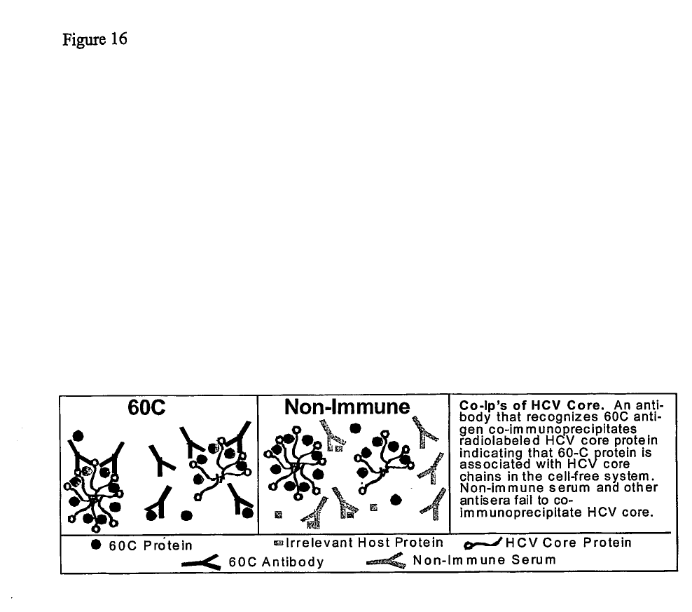

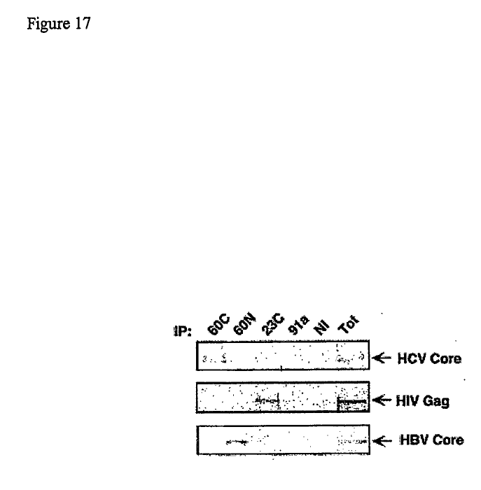

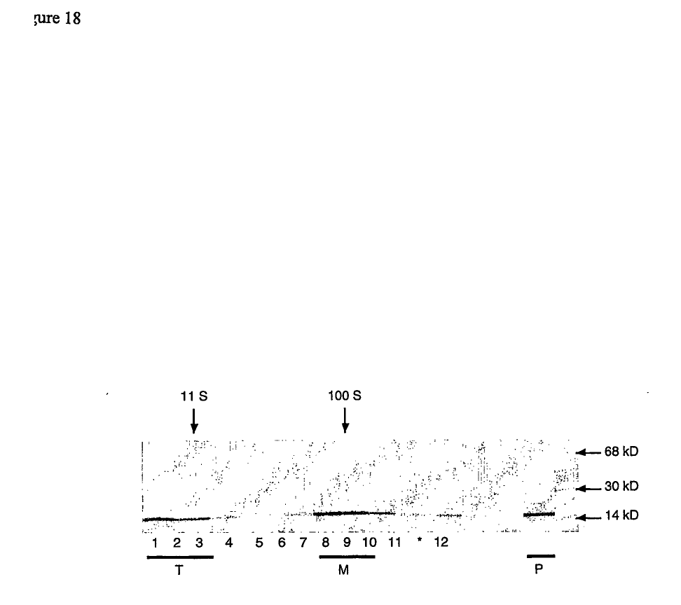

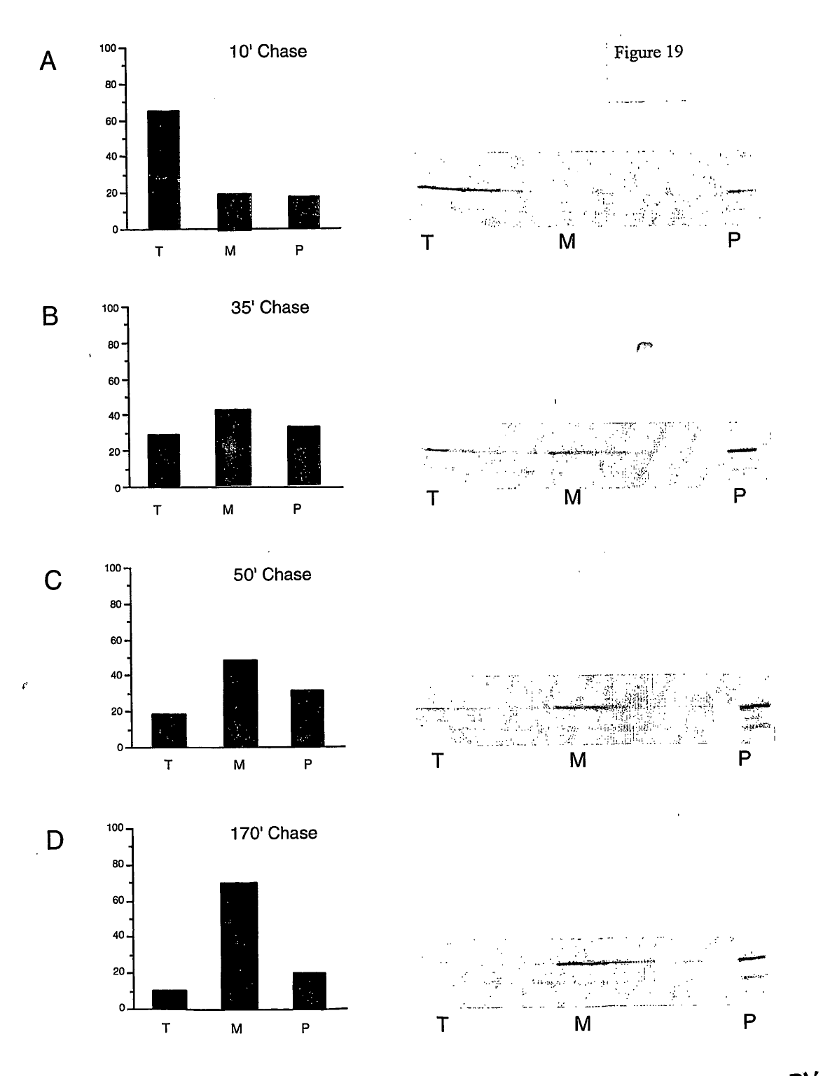

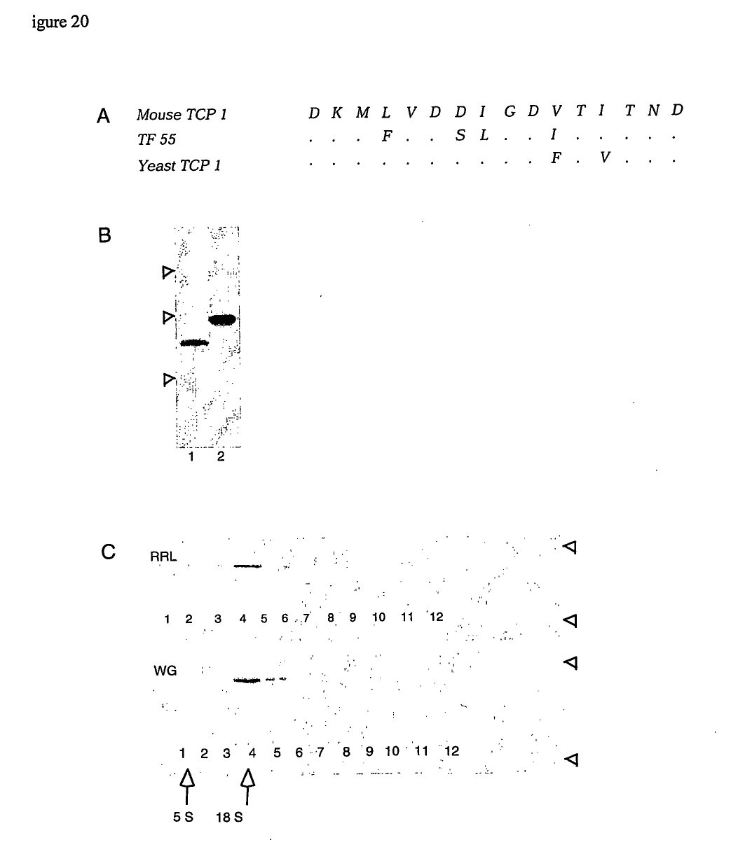

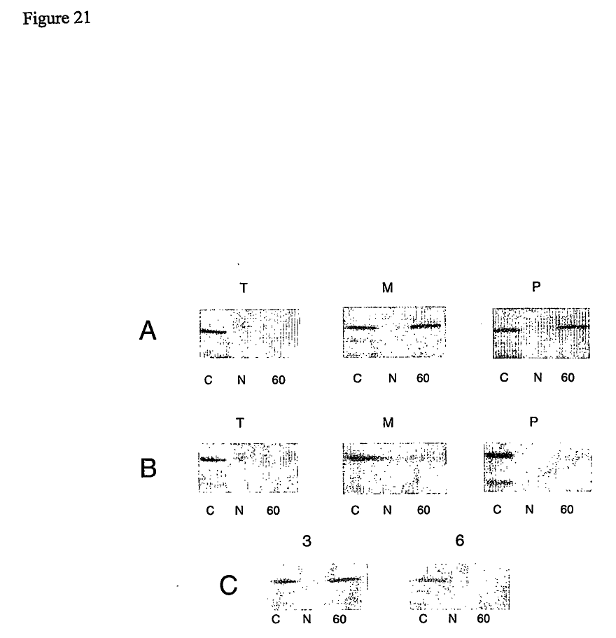

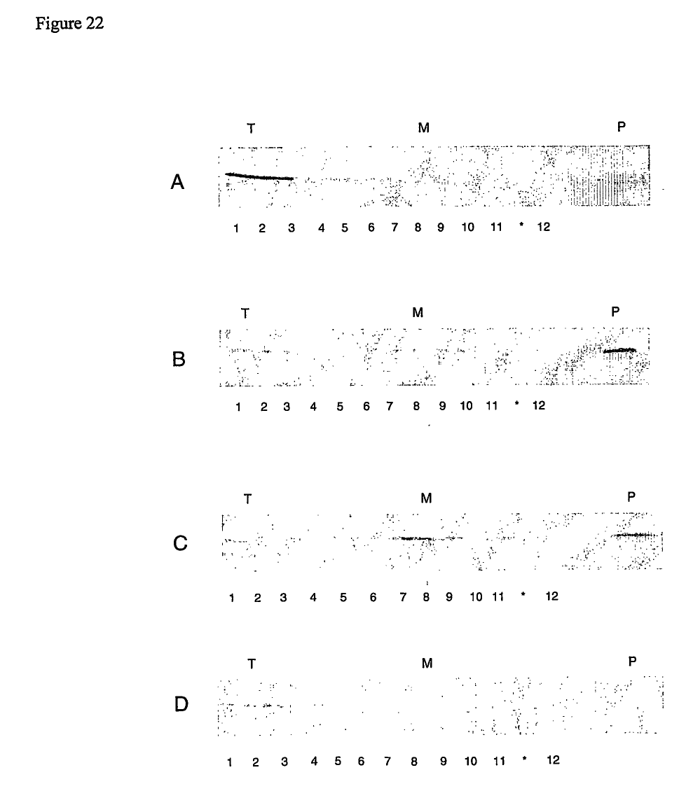

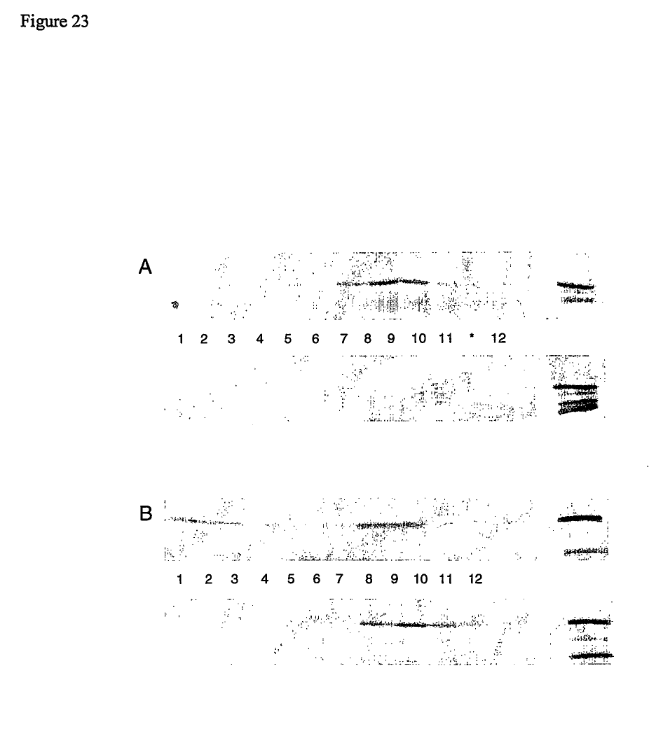

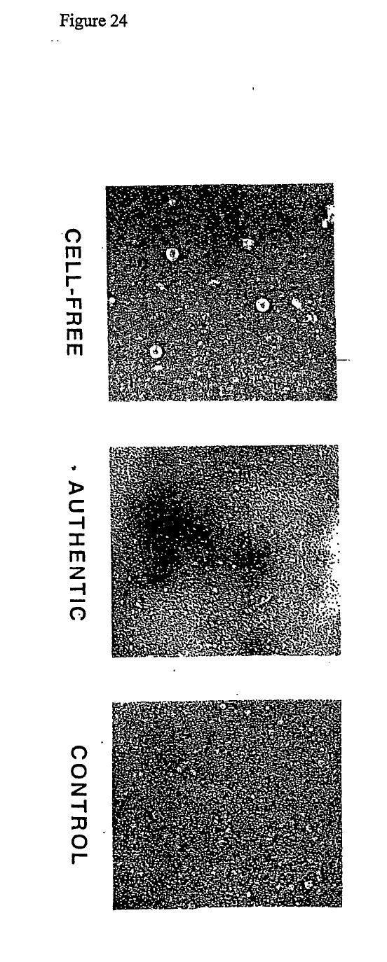

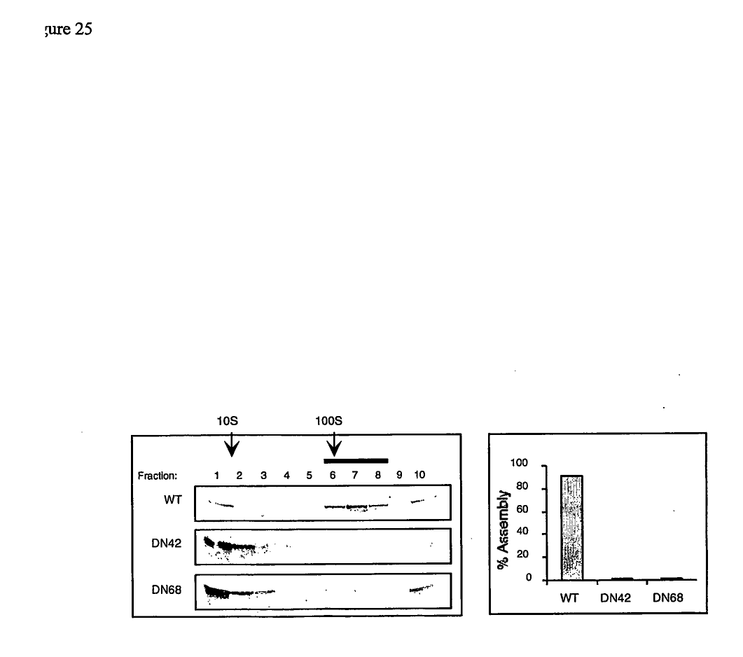

As molecular chaperones are likely candidates for promoting polypeptide assembly, antibodies directed against epitopes of various molecular chaperones were screened for their ability to co-immunoprecipitate radiolabeled Gag chains synthesized in the cell-free system. One one, the 23c monoclonal antibody (23c), co-immunoprecipitated radiolabeled Gag chains under native conditions (FIG. 4A), but not after denaturation which disrupted native protein-protein interactions (FIG. 4B). This antibody recognized a 3 amino acid epitope (LDD_{COOH}) present in several eukaryotic proteins, including the molecular chaperone TCP-1^{12,13}. 23c failed to co-immunoprecipitate other substrates translated in the cell-free system, including β-tubulin, α-globin, the Hepatitus B Virus capsid protein (core), and an assembly-incompetent mutant in Gag that is missing the NC and p6 domains (p41), under native conditions (FIG. 4A) or after denaturation (data not shown). Co-immunoprecipitation of HIV-1 Gag chains by 23c was inhibited in a dose-dependent manner by pre-incubating 23c with wheat germ (WG) extract (FIG. 4C). These data indicate that WG extract, which is used as the source of cytosolic factors for the cell-free assembly reaction, contains a protein recognized by 23c that selectively associates with assembling HIV-1 Gag chains.

23c recognized a single 68 kD WG protein both by immunoblotting (FIG. 4D) and by immunoprecipitation under native conditions (FIG. 4D, compare lanes 1 and 3). Velocity sedimentation of WG extract revealed that this 68 kD WG protein (WGH68 or HP68) migrated in a 5S fraction (data not shown). Its molecular weight and sedimentation characteristics indicated that HP68 does not correspond to either of the well-characterized proteins recognized by 23c, which include the molecular chaperone TCP-1 (a 55 kD protein that forms a 20S particle) and p105, a 105 kD component of the Golgi coatamer complex¹⁴. Recognition of P68 by Western blotting or immunoprecipitation was inhibited by addition of an LDD_{COOH}-containing peptide, but not a control peptide, at 125 μM (data not shown), indicating that 23c recognized HP68 by this epitope, as expected. To determine at what time during capsid assembly HP68 associated with Gag, a small cohort of Gag chains was radiolabeled by pulsing reactions with ³⁵S-cysteine and chasing with unlabeled cysteine. Co-immunoprecipitations were performed at different times during the pulse-chase assembly reaction. While the total amount of radiolabeled Gag in the reaction remained constant after the first 20 minutes (data not shwon), the amount of Gag co-immunoprecipitated by 23c increased over the course of the reaction to reach a peak at 120 minutes (FIG. 5A). These data suggest that SGHP68 associated with Gag not during Gag synthesis (which is largely completed by 45 minutes into the cell-free reaction⁷), but post-translationally, when Gag chains were forming multimeric complexes culminating in assembly of the completed immature HIV-1 capsid. The rapid drop in 23c immunoreactivity during the third hour of the assembly reaction (FIG. 5A) suggests that HP68 associated with Gag only transiently, releasing Gag chains once assembly was complete.

To determine whether HP68 was associated with specific assembly intermediates, a cell-free reaction programmed with Gag transcript was analyzed by velocity sedimentation and fractions were subjected to co-immunoprecipitation with 23c. Analysis of total products in these fractions revealed that radiolabeled Gag chains were present in the 750S completed immature capsid position (dark bar, FIG. 5B), as well as in the positions of the previously-described 10S, 80S, and 500S assembly intermediates. In contrast, Gag chains were co-immunoprecipitated by 23c only from the 10-80S and the 500S fractions (FIG. 5C). A gradient with higher resolution in the 10-80S range shows that the 80S intermediate, not the 10S intermediate, accounted for the majority of the 23c immunoreactivity in this range (data not shown). A cell-free Hepatitis B Virus capsid assembly reaction was analyzed in parallel as a control (data not shown). HBV core chains, which form both assembly intermediates and completely assembled capsids in the cell-free system¹⁵, were not co-immunoprecipitated by 23c. Thus, consisent with the results of the time course (FIG. 5A), analysis of Gag-containing complexes indicates that HP68 was selectively associated with partially-assembled, newly-synthesized HIV-1 Gag chains, nor with completely-assembled 750S capsids, nor with assembly intermediates of an unrelated virus.

### Example 6

### Purification, Sequencing, and Identification of HIV host Protein

For immunoaffinity purification, 1 ml WG extract was centrifuged at 100,000 rpm in a Beckman TL100.2 rotor for 15 min. The supernatant was subjected to immunoprecipitation using 50 μg of affinity purified 23c antibody (Stressgen) or an equivalent amount of control antibody (α-HSP 70, Affinity Reagents).

Immunoprecipitation eluates were separated by SDS-PAGE and transferred to a polyvinylidene difluoride membrane. A single 68 kD band was observed by Coomassie-staining in the 23c immunoprepicipation lane but not on the column. A portion of this band was excised for microsequencing (ProSeq, Salem, Mass.) and the remainder was used for immunoblotting to confirm that the band was recognized by the 23c antibody. The purified protein, which was blocked at the N-terminus, was cleaved with CNBr and treated with o-phthalaldehyde to allow selective microsequencing using Edman degeneration of peptides containing proline near the N-terminus.

The following degenerate 3′ oligonucleotides corresponding to the C-terminal peptide sequence of WGHP68 3′ was synthesized:

(SEQ ID NO.3)

ATGAATTC(ACTG)GG(ACTG)CG(GA)TA(GA)TT(ACTG)GT(ACTG)
GG(GA)TC
and

(SEQ ID NO.4)

ATGAATTC(ACTG)GG(CT)CT(GA)TA(GA)TT(ACTG)GT(ACTG)GG
(GA)TC.

The WGHP68 coding region was amplified by PCR using WG cDNA (Invitrogen), as the template, 3′ oligos corresponding to the WGHP68 C-terminal peptide sequence and 5′oligos corresponding to the vector into which the cDNA was cloned. This PCR reaction was performed four independent times and each time yielded a single 2 kB product. These PCR products were ligated into vectors by TA cloning (Invitrogen). DNA sequencing revealed each cDNA product to be identical. 3′ and 5′ coding and non-coding ends were obtained through nested RACE PCR reactions using degenerate oligos corresponding to sequences in the internal region of HP28. From overlapping cDNA clones, a complete open-reading frame for WGHP68 was defined. The start was identified by the presence of a defined Kozak consensus sequence at the initiating methionine, the presence of two in-frame stop codons upstream of the first methionine, the absence of ATG codons upstream from the presumptive start site (Kozak, Mamm Genome (1996) 7:563-74), and by homology to the human homologue in GenBank (Bisbal et al. J Biol Chem, (1995) 270:13308-17). The coding sequence for WGHP68 (SEQ ID NO: 5) has been deposited in GenBank under accession number AY059462.

Polyclonal rabbit antisera were generated against C-terminal peptides of Hu and WGHP68 (FIG. 6) and against the 19 N-terminal amino acids of human RNase L by injecting rabbits with peptides coupled to KLH. Affinity-purified αHuHP68b antisera was prepared by binding antisera to the HuHP68 C-terminal peptide coupled to agarose and eluting with glycine.

Cos-1 cells were transfected using Gag expression plasmids pCMVRev and PSVGagRRE-R described in Simon et al, J. Virology, (1997) 71:1013-18. HP68 plasmids for mammalian expression were constructed by using PCR to insert the coding regions for WGHP68, amino acids 1-378, Nhe1/Xba1 of pCDNA 3.1 (Invitrogen). Coding regions of all constructs were sequenced. Cells were transfected using Gibco Lipofectamine (Cos-1) or Lipofectamine Plus (293T). All transfections used a constant amount of DNA (18 μg per 60 mm dish). Medium was changed 24 hours after transfection and harvest was performed 28 or 60 hours after transfection for immunofluorescence and immunoblotting respectively. For immunofluorescence, cells were fixed in paraformaldehyde, permeabilized with 1% triton, and incubated with mouse HIV-1 Gag antibody (1:50) and affinity purified HuHP68 antiserum (1:2000), followed by Cy3- and Cy 2-coupled secondary (Jackson)(1:200). 178 cells were quantitated. For immunoblotting in FIG. 7 rat IgG was added to medium as a tracer at 10 μg/ml at the time of harvest, and cells were harvested in SDS sample buffer with boiling. For quantitation of immunoblotts, bands were compared to an immunoblot standard curve generated with known quantities of sample.

For immunoprecipitations followed by immunoblotting (FIG. 8 and 9), affinity purified α-HuHP68 antisera described above was coupled to Protein A beads (7 mg/ml beads) to generate αHuHP68b. Confluent Cos-1 cells in 60 mm dish were transfected, harvested in 300 μl NP40 buffer and 100 μl of lysine was immunprecipitated with 50 μl of αHuHP68b. Immunoprecipitates were analyzed by SDS-PAGE followed by immunoblotting with antibodies described.

WG extract (150 μl) was immunodepleted for 45 min at 4° C. with 100 μl beads coupled to antibody against WGHP68. Cell-free reactions (15 μl) were programmed (Lingappa et al., J. Cell Biol. 136:567-81 (1997)) using non-depleted WG or depleted WG. To some reactions containing depleted WG, purified WGHP68-GST or HuHP68-GST fusion protein or GST alone was added (2 μl of approx. 20 ng/μl) at the start of the reaction. After 3 hours at 26 ° C., NP40 was added to a final concentration of 1% and reactions underwent velocity sedimentation (5 ml, 15-60% sucrose gradients, Beckman MLS55 rotor: 45,000 rpm, 45 min). Thirty fractions, collected using a fractionator, were analyzed by SDS-PAGE and AR, followed by densitometry of Gag in each lane. For Proteinase K digestion, aliquots of fractions from the 500S and 750S regions of the gradient were collected and subjected to a 10 min incubation at RT with either no Proteinase K or 0.1 μg/ml Proteinase K. Digestion was terminated by adding SDS and freezing. Samples were analyzed by SDS-PAGE and AR. Graphs show average of three independent experiments (±SEM).

To generate purified HP68, WGHP68 and HuHP68 were subcloned into a pGEX vector (Pharmacia), to encode fusion proteins containing GST at the N-terminus. Expression was induced with 1 mM IPTG for 3 hours; sarcosyl (0.5%) and PMSF (0.75 mM) was added after sonication. 17,000×g supernatent was incubated with glutathione beads and eluted with 40 mM glutathione in 50 mM Tris, pH 8.0. Concentration of fusion protein and GST in eluate was determined using the Coomassie Plus protein assay Pierce).

Two cell-free reactions were programmed with HIV-1 Gag transcript and immunodepleted WG, and WGHP68-GST was added to one of these reactions. In parallel, Cos-1 cells were transfected resulting in expression of Gag and release of immature HIV-1 particles. The cell-free reactions and medium from transfected cells was treated with 1% NP40 to remove envelopes, and membranes associated with capsids, subjected to velocity sedimentation on 2 ml 20-66% sucrose gradients (Beckman TLS55 rotor, 35 min, 45,000 rpm).

Wheatgerm HP68 (WGHP68) was isolated from WG extracts by immunoaffinity purification using 23c antibody. Microsequencing yielded two well-defined sequences of 24 or more amino acids. Each sequence was approximately 70% homologous to-a different region of a single 68 kD protein identified as human RNase L inhibitor (Bisbal et al. JBC (1995) 270:13308-17; GenBank A57017, SEQ ID NO:6) (FIG. 6). Using degenerate oligonucleotides (SEQ ID NOs: 3 and 4) corresponding to the C-terminal peptide, a 2 kB cDNA was amplified from a WG cDNA mixture. Sequencing revealed that this cDNA had 70% identity overall to the cDNA coding for the 68 kD human RNase L inhibitor (here termed HuHP68) (Bisbal et al. JBC (1995) 270:13308-17; Bisbal et al. Methods Mol Biol (2001) 160:183-98). The open reading frame WGHP68 was deduced and its fill amino acid sequence was predicted (FIG. 6). The 604 amino acid sequence of WGHP68 shows 71% identity overall with the 599 amino acid sequence but of human RNAse L inhibitor (HuHP68). Both WGHP68 and HuHP68 contain two canonical ATP/GTP-binding motifs (Traut T. Eur J. Biochem (1994) 222:9-19) as well as the LDD-_{COOH} epitope (FIG. 6).

HuHP68 is known to bind and inhibit RNase L (Bisbal et al. JBC (1995) 270:13308-17; Bisbal et al. Methods Mol Biol (2001) 160:183-98), an interferon-dependent nuclease associated with polysomes (Salehzada. et al JBC (1991) 266:5808-13; Zhou et al. Cell (1993) 72:753-65) and activated by the interferon-sensitive 2′-5′ linked oligoadenylate (2-5S) pathway. Interferon-dependent induction and activation of RNase L results in degradation of many viral RNAs (Player et al. Pharmacol Ther. (1998) 78:55-113; Samuel C. Virology (1991) 183:1-1 1; Sen et al. JBC (1992) 267:5017-20). Previously, overexpression of the 68 kD RNAse L inhibitor (HuHP68) in HIV-1-infected cells has been shown to increase virion production by reducing RNase L activity, resulting in higher levels of HIV-1 RNA and HIV-1 specific protein (Martinand et al. J. Virology (1999) 73:290-6). These findings that WGHP68 binds to Gag-containing, post-translational intermediates during cell-free HIV-1 capsid assembly led to further investigation of whether HuHP68 binds to and acts on fully-synthesized Gag chains post-translationally in cells, in addition to binding and inhibiting RNase L as previously described (Salehzada et al. JBC (1991) 266:5808-13; Zhou et al. Cell (1993) 72:753-65).

### Example 7

### Association of HP68 with HIV-1 Gag infected Human Cells

To analyze the function of HP68 in cells, a peptide-specific polyclonal antibody was generated against both internal and C-terminal residues of WGHP68, and C-terminal residues of HuHP68. These antisera specifically recognized a 68 kD protein in WG and in primate cells respectively, by immunoporecipitation as well as Western blotting. Detection of the 68 kD band was eliminated by pre-incubating each antibody with the peptide against which it was directed. Affinity-purified antisera to HuHP68 were generated and coupled to Protein A beads (αHuHP68b), and found to have a high affinity for both human and simian HP68. To determine whether HP68 was associated with assembling HIV-1 Gag chains in human cells, human 293T cells were transfected with a plasmid (pBRUΔenv) encoding the entire HIV-1 genome except for a deleted portion of the env gene²⁵. Cells were harvested in non-ionic detergent and subjected to immunoprecipitation under native conditions and after denaturation using αHuHP68b. Immunoprecipitates were analyzed by Western blotting using a monoclonal antibody to HIV-1 Gag. HIV-1 Gag was co-immunoprecipitated by αHuHP68 under native condition but not after denaturation (FIG. 8A). HP68 appeared to associate with Gag post-translationally. These data revealed that HuHP68 was associated with HIV-1 Gag in human cells that were producing mature HIV-1 virions.

Further investigation revealed that HP68 was associated with Gag in RNase-treated and unteated cell lysates analyzed in parallel (FIG. 8A). These findings that HuHP68 bound completely-synthesized Gag chains, and did so in the absence of intact RNA, indicated that this host protein was bound to Gag-containing complexes post-translationally. As shown in FIG. 8B, Gag was associated with HP68 under native conditions, but not after denaturation when immunoprecipitated with αHuHP68b. This confirmed that HP68 bound to HIV-1 Gag in the absence of the HIV-1 protease and other HIV-1 specific proteins. As shown in FIG. 8C HP68 was associated with wild-type Gag and with the assembly-competent p46 mutant; but was not associated with an assembly incompetent p41 mutant. Thus, HP68 associated specifically with assembling Gag chains in mammalian cells, as it did in the cell-free system. To confirm that HP68 associates with Gag in HIV-1-infected cells, co-immunopecipitations on lysates of human T-cells producing fully infectious HIV-1 (ACH-2 cells) were performed. Anti-HuHP68 co-immunoprecipitates HIV-1 Gag chains under native conditions but not after denaturation in phorbol myrisate acetate (PMA)-stimulated, chronically-infected ACH-2 cells, which release high levels of infectious HIV-1. The same results were observed with unstimulated ACH-2 cells (data not shwon), which produce low levels of infectious virus.

Confirmation of co-association of HP68 and Gag was demonstrated using immunofluroescent microscopy of Cos-1 cells transfected with the HIV-1 expression vector pBRU Δenv (FIG. 10, columns 1-3) and double-labeled with antibodies to HIV-1 Gag and HP68. HIV-1 Gag was expressed in approximately 40% of cells, and was found in a predominantly clustered pattern (FIG. 10B, E, and H) that likely represents localization of Gag to sites of particle formation and budding at the plasma membrane³². HP68 staining revealed two different patterns of localization. HP68 was present in a diffuse pattern in 100% of the cells that failed to become transfected and did not express HIV-1 Gag (two cells on left in FIG. 10A-C), as well as in 100% of control cells that were transfected with constructs expressing control proteins. In cells expressing HIV Gag, HP68 was also found in a coarsly clustered pattern (FIG. 10D and G). FIG. 10C, F and I show a merged image where there was a striking co-localizaion of HP68 and Gag in the yellow coarse clusters. Recruitment of HP68 into clusters containg Gag was seen in 100% of cells that were expressing HIV-1 Gag. In contrast, when cells were transfected with pBRUp4Δenv, which encodes an assembly defective truncation of Gag (p41), HP68 was not found in a clustered pattern or co-localized with HIV Gag (FIG. 10G-I).

### Example 8

### The HP68 Gag Complex Selectively Associates with HIV-1 Vif but not with RNase L

The purpose of this experiment was to determine if the post-translational HP68-Gag containing complex involved in virion formation is distinct from the post-transcriptional RNase L-containing complex, even though both contain HP68.

Cos-1 cells expressing pBRUΔenv were subjectedf to immunoprecipitation using αHuHP68b followed by immunoblotting with antibodies to Vif and Nef to determine whether either of these viral proteins are present in the HP68 complex. Imnunoblotting was also performed with antibodies to the cellular proteins RNase L and actin. αHuHP68b co-immunoprecipitated Gag and Vif from cells under native conditions but only immunoprecipitated HIV-1 Gag under native conditions (FIG. 11A). In addition, long exposures revealed that in cells expressing pBRUΔenv αHuHP68b co-immunoprecipitated full-length GagPol, which should be present along with Gag in assembling virions (data not shown). HIV-1 Vif, which is involved in virion assembly, was also co-immunoprecipitated under native but not denaturing conditions. In contrast, HIV-1 Nef, a viral protein that is likekly incorporated into the virion through a direct association with the plasma membrane, was not found associated with HP68, indicating that only selected HIV-1 proteins are associated with HP68. The association of Gag and Vif with HP68 was present even when cells were lysed in 10 mM EDTA (FIG. 11B). Further evidence for the specificity of the HP68-Vif interaction was obtained by demonstrating that the abundant cellular protein actin was not associated with the HP68 (FIG. 11A). Finally, RNase L was not present in the HP68-Gag-Vif-containing complex, supportin the idea that this complex is distinct from the previously described RNAse L-HP68 complex (FIG. 11A). To confirm the specificity of the Vif association, co-immunoprecipitation with αHuHP68b was performed in the presence of the HuHP68 C-terminal peptide that was used to generate αHuHP68b (FIG. 6). At 200 μM, the HuHP68 peptide, which binds specifically to αHuHP68, blocks immunoprecipitation of HP68, Gag, and Vif by αHuHP68b (FIG. 11B). Thus, the HP68-Gag containing complex is specifically associated with a second HIV-1 viral protein, Vif, but does not contain either RNAse L or an abundant non-specific cellular protein (actin) and is unaltered when ribosomes are disrupted. All of these findings argue that HP68 has a post-translational function that is separated from its post-transcriptional action as an RNAse L inhibitor.

### Example 9

### HP68-Gag Also Binds to Pol

To further demonstrate that HP68 has a second function during viral assembly, we show that the post-translational HP68-Gag complex does not contain RNase L, but does contain two other viral proteins known to be involved in virion morphogenesis, namely, HIV-1 GagPol and HIV-1 Vif (FIG. 11). The selective association of HP68 with 3 proteins that are critical for assembly of a fully-infectious virion (Gag, GagPol, and Vif) provides strong support to the functional data demonstrating an essential role for HP68 in capsid formation. In particular, the association fo HIV-1 Vif with HP68 underscores the importance of HP68 in virion formation, since HIV-1 is required for formation of virions that are fully infectious for cells that are natural targets of infection in vivo. Furthermore, Vif is known to act by an undefined mechanism on virion assembly in producer cells, and is very likely to require interaction with an as yet unidentified host factor that is critical for its function. Binding of Vif to HP68 appears to be independent of HIV-1 Gag, since it occurs when HIV- 1 Gag is expressed as a mutant truncated proximal to NC) that fails to bind to HP68. Thus, HP68 acts in a complex with at least 3 proteins involved in virion assembly. This complex (or complexes) plays a critical post-translational role in virion formation and is separate from the previously described RNase L-HP68 complex that protects viral mRNA from host-mediated degradation post-transcriptionally. It is possible that Vif binds to HP68 in more than one different assembly intermediate complex, as is the case for Gag.

### Example 10

### The Viral-Host Interaction is Conserved Among Primate Lentiviruses

The importance of the post-translational role of HP68 in the retroviral life cycle is further underscored by the observation that both HIV-2 and SIV mac239 Gag bind to HP68, indicating conservation of this viral-host association among primate lentiviruses (see FIG. 12).

### Example 11

### Assembly of HCV Capsid in a Cell-Free System

Wheat germ extracts are used to program the translation and assembly of HCV core polypeptides in a manner analogous to the HIV capsid assembly system as described in Example 1, with the exception that it is not necessary to add myristoyl CoA to the system. In order to support efficient immature HCV-1 capsid assembly the extracts were ultracentrifuged briefly (most likely to remove an inhibitor; see Lingappa, et al., (1997) Cell Biol 136:-567-81). HCV capsid-assembly does-not appear to be affected by addition of non-ionic detergent to the assembly reactions. This is consistent with the idea that HCV core probably assembles into pre-formed capsids in the cytoplasm. While HCV core has been shown to have a hydrophobic tail that is associated with the cytoplasmic face of the ER membrane (Santolini, et al., (1994) J Virol 68-3631-41; Lo, et al., (1996) J Virol 70: 5177-82). This association apparently is not required for proper HCV capsid assembly, and may instead play a role in association of HCV core with the E1 envelope protein.

After incubation for 2.5 hours, the products of the HCV core cell-free reaction were analyzed by velocity sedimentation on 2 ml sucrose gradients containing 1% NP40 (55,000 rpm×60 min. in Beckman TLS55 rotor). Fractions (200 microliters each) were collected from the top of gradient and examined by SDS-PAGE and autoradiography. A particle of ˜100S was produced in this reaction (see FIG. 13). Thirty to 50% of newly-synthesized HCV core chains form these ˜100 S particles by the end of the reaction, located in the middle (M). The remainder of HCV core chains are in the top fraction (T) and in the pellet (P) closely resembling what we have seen previously with assembly of HBV core into capsids in a cell-free system (Lingapaa, J. R., et al., (1994) J Cell Biol 125: 99-111). To confirm that the 100S de-enveloped particle represents HCV capsids, the factions containing de-enveloped capsids (lanes 6 and 7) from the velocity sedimentation gradient were analyzed by equilibrium centrifugation on CsC1 (50,000 rpm×20 hours using a TLS55 Beckman rotor) using a 337 mg/ml CsC1 solution. Fractions were collected, TCA precipitated, analyzed by SDS-PAGE and autoradiography, and quantitated by densitometry. HCV core protein peaked in fraction 6. The density of fraction ⅚ (middle of the gradient, indicated with arrow) is 1.25 g/ml. The buoyant density of approximately 1.25 g/ml (FIG. 14), is identical to that of HCV capsids (without envelopes) produced in infected cells (Kaito, M. et al., ((1994) J Gen Virol 75: 1755-60; Miyamoto, H. et al., (1992) J Gen Virol 73: 715-8).

Fractions containing the 100S particle were analyzed by transmission EM [(TEM)/Fractions 6 and 7 from the velocity sedimentation gradient described in FIG. 14 were pooled, put on a formvarcoated grid, negatively-stained with uranyl acetate, and examined by TEM.] 30-50 nm spherical particles composed of capsomeric subunits were clearly seen. This is the size expected for HCV capsids that have had their envelopes removed or that are not yet enveloped (Mizuno, et. al., (1995) Gastroenterology 109: 1933-40; Takahashi, et al., (1992) Virology 191: 431-4). (Note, in contrast, that ribosomes have a diameter of 12-20 nm.) Thus, by three criteria presented here (velocity sedimentation, buoyant density, and electron microscopy), HCV forms capsids in the cell-free system that closely resemble those found in infected cells.

### Example 12

### Assembly of HCV Core Truncations Containing the Homotypic Interaction Domain

Previous findings indicate that the HCV core interaction domain is located in the hydrophilic region from aa 1 to 115 (Matsumoto, et al., (1996) Virology 218:43-51; Nolandt, O. et al., (1997) J Gen Virol 78: 1331-40; Yan, B. B., et al., (1998) Eur J Biochem 258:100-6;Kunkel, M. et al, (2001) J Virol 75: 2119-29). Therefore HCV core truncations that encompass this domain should assemble into completed capsids in the cell-free system. Assembly reactions were programmed with transcripts encoding C191, C115, and C124. Total synthesis was similar for all 3 constructs. After incubation for 2.5 hours reaction products were analyzed by velocity sedimentation, and the amount of core that migrated in 100S particles was graphed as % of total core synthesized (FIG. 15 below). The two C-terminal truncation mutants assembled into 100S particles, as did full-length core. The finding that the domains required for assembly are located in the first 115 amino acids of HCV core is consistent with observations in other systems (Matsumoto, et al., (1996) Virology 218:43-51).

Mutants of HCV core were also engineered to encode amino acids 42-173 (ΔN42) and amino acids 68-173 (ΔN68) (FIG. 25). Transcripts of wild-type (WT) C173 core (amino acids 1-173) or the N-terminal deletion mutants described above were used to program cell-free translation and assembly reactions. Reaction products were analyzed by velocity sedimentation on sucrose gradients. In FIG. 25, left panel, cell-free assembly reactions were programmed with WT and mutant core transcripts and separated by velocity sedimentation. Fractions were analyzed by SDS-PAGE and autoradiography. S values are indicated above fractions, and the dark bar indicates the expected position or fully assembled capsids. FIG. 25, right panel, shows the amount of radiolabeled core that migrates in the 100S fraction as a percentage of total core protein synthesized (% assembly), which was determined by densitometry of autoradiographs in the left panel. Wild-type C173 assembled into 100S capsid-like structures very efficiently.

### Example 13

### Evidence that HCV Capsid Assembly Proceeds Through an Ordered Pathway of Intermediates

To determine whether capsid assembly occurs by way of assembly intermediates, a pulse-chase experiment was performed in the cell-free system. Cell-free reactions were programmed with wild-type HCV core, labeled for 3 min. with 35-S cysteine, and chased with unlabeled cysteine. Aliquots were taken at the times indicated, and analyzed by velocity sedimentation on 2 ml sucrose gradients, as described in FIG. 15. Fractions were examined by SDS-PAGE, and autoradiographs were quantitated. The graph shows amount of HCV core protein present in the top fractions 1 and 2 (T), vs. middle fractions 6, 7, and 8 (M), vs. pellet (P). Middle fractions represent 100S completed HCV capsids. Progression of labeled core polypeptides through complexes of different sizes was examined by velocity sedimentation of aliquots taken at different times during the chase reaction. The results suggest that capsid proteins first appear at the top of the gradient (˜10-20S complexes that are likely to represent dimers or small oligomers), then appear in the pellet, which may represent a large assembly intermediate, and finally appear in the middle of the gradient (˜100 S), in the position of completed capsids. These results indicate capsid assembly occurs through an ordered pathway of assembly intermediate complexes. The pellet increases initially, and then decreases as completed capsids are formed, indicating the presence of a high-molecular weight assembly intermediate in the pellet.

### Example 14

### HCV Core Proteins Appear to be Associated with a Host Protein in the Cell-Free System.

Studies of other viral capsids such as HIV-1 and HBV capsids (see above), suggest that capsid assembly in cells is energy-dependent and requires host factors (Lingappa, J. R., et al., (1997) J. Cell Biol 136:567-81; Lingappa, J. R. (1994) J Cell Biol 125: 99-111; Weldon, R. A., et al., (1998) J Virol 72: 3098-106; Mariani, R., et al., (2000) J Virol 74: 3859-70; Mariani, R. et al., (2001) J Virol 75: 3141-51; Unutmaz, D., et al., (1998) Sem in Immunol 10: 225-36). Cellular factors are also implicated in HCV capsid assembly, since assembly of full-length core in the absence of cellular factors results in particles that have abnormal sizes and shapes as compared to capsids produced in cells (Kunkel, M., et al., (2001) J Virol 75: 2119-29).

Using the cell-free system to search for host factors that could be involved in capsid formation, two assumptions were made: 1) that such a host factor likely is associated with core chains transiently during assembly, and 2) that candidates for host factors involved in HCV capsid assembly include the general class of molecular chaperones, in particular eukaryotic cytosolic chaperones. Proteins that are recognized by antibodies directed against the eukaryotic cytosolic chaperone TCP-1 have been found associated with capsid proteins of two different viruses, namely HBV (Lingappa, J. R. (1994) J Cell Biol 125: 99-111). And the type d retrovirus Mason-Pfizer Monkey Virus (M-MPV) Hong, S., et al, (2001) J Virol 75: 2526-34). Note that in both of these studies, TCP-1 has not been definitively identified as the co-associating protein, so the possibility of a cross-reacting protein has not yet been ruled out. The capsids of both of these viruses pre-form in the cytoplasm, unlike the capsids of type C retroviruses such as HIV -1 (Wills and Craven (1991) Aids 5: 639-54).

To look for an association of HCV core with molecular chaperones, cell-free reactions were programmed with either HCV core, HIV-1 Gag, or HBV Core. During assembly, reactions were subjected to immunoprecipitation (IP) under native conditions with antisera directed against different epitopes of TCP-1 (60-C, 60-N, 23c, and 91a) or with non-immune serum (NI). IP eluates were analyzed by SDS-PAGE and autoradiography. All of the antibodies tested failed to recognize HCV core chains in these assembly reactions except one, suggesting that most molecular chaperones are not associated with assembling full-length chains of HCV core. However, an antiserum (60-C) directed against a specific epitope (aa 400 to 422) of the eukaryotic cytosolic chaperonin TCP-1 co-imnmunoprecipitated HCV core under native conditions (FIG. 16 and 17). These data suggest that either TCP-1 or a protein that shares an epitope with TCP-1 is associated with HCV core chains in the cell-free system. The epitope recognized by this antiserum corresponds to the sequence:
N-terminus—RGANDFMCDEMERSLHDA—C-terminus
   This epitope is highly conserved among TCP-1 isolated from different species. In addition, this epitope has sequence homology to a region of the bacterial chaperonin GroEL. In general, GroEL shares little overall sequence specificity with TCP-1, but has a very similar structure and function (Frydham, J. et al., (1992) Embo J 11: 4767-78;Gao, Y. et al., (1992) Cell 69: 1043-50; Lewis, V. A., et al., (1992) Nature 358: 249-52;Rommelaere, H. et al., (1993) Proc Natl Acad Sci USA 90: 11975-9;Yaffe, M. B. et al., (1992) Nature 358: 245-8). A BLAST search using the 60-C sequence does not reveal any other proteins having significant sequence homology to the 60-C sequence besides TCP-1 subunits from various species.

Antisera directed against other regions of TCP-1, such as 60-N(Lingappa, J. R., et al., (1994) J Cell Biol 125: 99-111), 23c (Hynes, G. et al., (1996) Electrophoresis 17: 1720-7; Willison, K et al, (1989) Cell 57: 621-32), and 91a (Frydman, J. et al., (1992) Embo J 11: 4767-78), fail to co-immunoprecipitate HCV core. In contrast, HBV core is recognized by the 60-N antiserum (directed against aa 42-57 in TCP-l (Lingappa, J. R. et al., (1994) J Cell Biol 125: 99-111). Assembling chains of HIV Gag are recognized by the 23c antiserum (which recognizes an epitope containing the last 3 amino acids in TCP-1) (Lingappa, J. R. et al., (1997) J Cell Biol 136: 567-81), as shown in FIG. 17. These differences in epitope recognition-are-consistent with the possibility that each of these capsid proteins binds to a different host protein. Alternatively, if capsid proteins of two unrelated viruses bind to the same cellular protein (which may be the case for HBV and HCV core), one would expect that each would bind to that protein in a unique way, since capsid proteins of unrelated viruses have no significant sequence homology to each other. Thus, different epitopes are like to be exposed when two unrelated capsid proteins bind to the same cellular protein. Together, the data strongly argue that capsid proteins of different viruses form unique interactions with host proteins during assembly.

### Example 15

### HBV Core Cell-free Translation Products Migrate in Three Positions upon Velocity Sedimentation

To synthesize radiolabeled HBV core polypeptides, HBV core DNA was transcribed in vitro and translated for 120 min in a heterologous cell-free system contain wheat germ extract (see Example 1). The radiolabeled translation products were analyzed for formation of HBV core multimers by sedimentation on 10-50% sucrose gradients at 200,000 g for 1 h. Following fractionation of the gradients, the migration of radiolabeled core proteins was determined using SDS-PAGE, Coomassie staining, and autoradiography. Under these conditions, unlabeled protein standards of less than 12 S, such as catalase, migrated in the first three fractions. Mature core particles produced in recominant E. coli (referred to as authentic capsids) were found predominantly in fractions 5-7 (˜100 S). Radiolabeled cell-free translation products were found to migrate in three distinct positions usin these gradient conditions, as shown in FIG. 18. The first region, at the top of the gradient (7) corresponds to the position of monomeric and small oligomeric core polypeptides, while the second region, in the middle of the gradient (M), corresponds to the position of authentic capsids. The third region, in the pellet (P), represents very high molecular weight structures. The possibility that either the pellet or the middle fraction consists of completed chains not yet released from ribosomes was ruled out by treatment of the translation products after completion of synthesis with EDTA, which is known to disassemble ribosomes (Sabatini et al., 1966). Both pellet and middle fractions were largely unaffected by EDTA treatment (data not shown). Taken together, these results raised the possibility that capsid-like particles were being assembled from newly synthesized core polypeptides in this cell-free system.

To confirm the authenticity of the capsids produced in the cell-free system, relevant fractions were examined by EM. The products of cell-free translation of HBV core (FIG. 24, Cell-Free) and of cell-free translation of an unrelated protein (GRP 94) (FIG. 24 Control) as well as recombinant HBV capsids (FIG. 24, authentic) were treated with EDTA to disassemble ribosomes and then centrifuged to equilibrium on CsC1 gradients. Fractions 6 and 7 of each of these gradients were collected and concentrated in an Airfuge. Electron micrographs of the resuspended pellets examined by a microscopist in single blinded fashion revealed particles indistinguishable from authentic capsids in the products of HBV core cell-free translation. In contrast, no particles resembling capsids were seen in the equivalent fractions of the cell-free translation of an unrelated protein. Thus, by four criteria—velocity sedimentation, buoyant density, protease resistance (data not shown), and electron microscopy—a portion of the HBV core translation products assembles into bona fide HBV capsids.

To determine the order of appearance of labeled core polypeptides in top, middle, and pellet fractions of the sucrose gradient described in FIG. 18, cell-free translations were performed using a 10-min pulse of [³⁵S]cysteine, followed by a chase for varying lengths of time in the presence of excess unlabeled cysteine. Translation products were sedimented through sucrose gradients and analyzed by SDS-PAGE and autoradiography. After a 10-min chase period, a time at which essentially all of the labeled chains have completed translation, the cohort of chains synthesized in the presence of labeled cystein was found predominantly in the top of the gradient (FIG. 19A). Upon extending the chase period to 35 min, a significant amount of material was found in both the pellet and the middle of the gradient (FIG. 19B). Following a chase period of 50 min, there were very few labeled chains present at the top of the gradient. Rather, increasing amounts of label had accumulated in he pellet and middle fractions (FIG. 19C). After a 170-min chase period, the amount of radiolabeled material in the middle underwent a further increase with a decrease in labeled material in both the pellet and top fractions (FIG. 19D). Quantitation of autoradiographs, shown next to the corresponding gels, confirmed that the labeled material at the top of the gradient diminished dramatically over time. The material in the pellet initially increased and then decreased, while the material in the middle accumulated progressively over the course of the chase period. Thus, the data indicate that newly synthesized core polypeptides chase over time into HBV capsids, and it is likely that they do so, at least in part, by way of a high molecular weight complex contained within the pellet. Definitive confirmation that the pellet contains an intermediate in the formation of completed capsids is shown in FIG. 23.

### Example 16

### CC 60 is Associated with Intermediates in the Assembly of HBV Capsids

A polyclonal rabbit antiserum (anti 60) was raised against a peptide sequence of TCP-1 (FIG. 20A). Studies by others have shown that TCP-1 is a protein of ˜60 kD that migrates as a so-S particle (Gao et a., 1992; Yaffe et al., 1992). From total extracts of steady state-labeled HeLa cells, our anti 60 antiserum immunoprecipitated a single 60-kD protein under denaturing conditions (FIG. 19B, lane 1). The same 60-kD protein was immunoprecipitated by anti 60 under native conditions (Martin, R., and W. J. Welch, manuscript in preparation). When either rabbit reticulocyte lysate or wheat germ extract was fractionated on a 10-50% sucrose gradient, the ant 60-reactive material migrated as a 20-S particle as revealed by imunoblotting of gradient fractions (FIG. 20C, top and bottom, respectively). Furthermore, a 60-kD polypeptide component of a 20-S particle (purified from reticulocyte lysate) that is known to be recognized by a previously described antibody to TCP-1 (Willison et al., 1989) also reacted to the anti 60 antisera described here (H Sternlicht, personal communication). Mitochondrial hsp 60, in conrast, failed to be recognized by anti 60 (data not shown). The 20-S particle recognized by anti 60 also was recognized by an antibody provided by J. Trent, Argonne National Laboratory, Argonne, Ill.)(see Trent et al., 1991) against TF 55, the hsp 60 homolog found in the thermophilic archaebacterium Sulfolobus shibatae (data not shown). Thus, anti 60 appears to be recognizing either TCP-1 or a closely related eukaryotic cytosolic protein, which we refer to as C60.

To determine whether CC 60 is associated with HBV core in the cell-free assembly system, and whether anti 60 (FIG. 21, 60) was able to coprecipitate newly synthesized HBV core polypeptides from various fractions of the sucrose gradients was examined. Control immunoporecipitations were performed using nonimmune serium (FIG. 21, N) as well as polyclonal rabbit antiserum to HBV core polypeptide (FIG. 21 C). FIG. 21A shows that under native conditions and 60 coprecipitated radiolabeled core polypeptides present within the middle (M) and the pellet (P) of the sucrose gradients, but did not coprecipitate core polypeptides from the top (T). Similarly, antibody to TF 55 (see above) coprecipitated core polypeptides in the pellet and the middle of the gradients (data not shown). As expected, when immunoprecipitations were performed after denaturation of samples by boiling in SDS, anti 60 no-longer-coprecipitated core polypeptides from any of these gradient fractions (FIG. 21B). In contrast, antiserium to core polypeptide recognized labeled core protein in all three of these fractions under both native and denaturing conditions (FIG. 21, A and B). Based on these observations, it appears that CC 60 is not associated with unassembled forms of HBV core protein, but is associated with multimeric forms of the protein. These results raised the possibility that CC 60 plays a role in the assembly of HBV core particles.

If CC 60 were to play a role in assembly, one might expect this chaperonin to dissociate from the multimeric core particle once assembly is complete. To test this hypothesis we performed immunoprecipitations on material from the middle of sucrose gradients that had been further fractionated on a CsC1 gradient. Using such an equilibrium centrifugation method we can separate mature capsids (found in fractions 1-4 of the CSC1 gradients) and are possibly incomplete assembly intermediates. FIG. 21 C shows that under native conditions, anti 60 precipitates HBV core polypeptides present in fraction 3 from CsC1 gradients (corresponding to incomplete capsids) but fails to precipitate core polypeptides present in fraction 6 from the same gradients (corresponding to completed capsids). Antiserum to core polypeptide recognizes core protein in both fractions. Thus it appears that CC 60 is associated with partially assembled capsids, but is not associated with mature capsids.

As further confirmation that CC is only transiently associated with core polypeptides in the process of assembly, immunoblots of gradient fractions were performed with antiserum to CC 60 at different times during translation (Lingappa, J. R., W. J. Welch, and V. R. Lingappa, manuscript in preparation). These immunoblots revealed the presence of a large amount of CC 60 in the pellet at early time points during ranslation of HBV core transcript but not during translation of mock transcript. In contrast, at later times during the core translation and assembly reaction, all of the CC 60 was located in the 20-S position with none remaining in the pellet. In these experiments the total amount of CC 60 was essentially unchanged over the course of translation.

### Example 17

### HBV Core Polypeptide Production Can Be Uncoupled from Core Particle Assembly

To distinguish between a role for CC 60 in-folding of core monomers versus a role in assembly of multimeters, we attempted to uncouple production of core polypeptides from core particle assembly. In Xenopus oocytes, assembly of core particles is known to be exquisitely dependent on the concentration of core polypeptide chains (Seifer et al., 1993). We observed an equally striking concentration dependence in our system. When we decreased the concentration of HBV core transcript to 50% or less of the standard concentration used in our cell-free system, HBV capsid assembly was virtually abolished (FIG. 22A), while total core polypeptide synthesis was diminished in a roughly linear fashion (data not shown). These conditions resulted in the accumulation of a population of unassembled, full-length core polypeptides that migrated at the top of the previously described sucrose gradients (FIG. 22A). Even when incubated for a long time (6 h), these unassembled chains remained at the top of the gradient indicating that asembly does not occur even at a slow rate under these conditions (data not shown), When centrifuged on a 5-25% glycerol gradient for 14 h, the unassembled core polypeptides migrated in the approximate region expected for folded globular dimers of core, based on the position of protein standards (data not shown). Thus, the data indicate that the unassembled material at the top of the gradient does not consist of unfolfed polypeptides. Rather, this material likely represents core polypeptide dimers, or a mixture of monomers and dimers. Dimers are known to be capsid assembly precursers in vivo (Zhou and Standring, 1992).

To determine whether the unassembled core polypeptides present at the top of the gradient are in fact competent for assembly into capsids, we asked if they could be chased into capsids in the presence of excess unlabeled core chains. To do this we added to these unassembled radiolabeled chains an excess of an unlabeled translation mix that had been programmed with 100% core transcript for 45 min. The 45-min time point was chosen because it represents a point at which the newly synthesized core chains are present in roughly equal proportions in the top, middle, and pellet regions of our standard sucrose gradients (data not shown). After mixing the labeled, unassembled chains with the unlabeled translation, incubation was continued at 24° C. for either 45 or 120 min and the mixture was then layered onto sucrose gradients, centrifuged, fractionated, and analyzed by SDS-PAGE and autoradiography as previously described. After a 45-min incubation, the labeled polypeptides were found primarily in the pellet (P) with a small amount in the middle of the gradient (M) (FIG. 22B), while after 120 mins a significant quantity of labeled chains was present in the middle of the gradient (FIG. 22C). When material from the middle of that sucrose gradient (FIG. 22C) was subsequently centrifuged on CsC1, the radiolabeled chains were found to comigrate with authentic core particles confirming that completed capsids were produced during the chase (data not shown).

When an unlabeled mock translation was preincubated for 45 min and added to the unassembled core polypeptides, the radiolabeled core polypeptides at the top of the gradient failed to chase into either the pellet or the middle (FIG. 22D). A similar result was obtained when a translation programmed with bovine prolactin, an unrelated protein, was added to the unassembled core polypeptides. Likewise, when an unlabeled translation of 50% of the standard core transcript was added to the unassembled radiolabeled core polypeptides, the radiolabeled chains remained at the top of the gradient (data not shown). In the latter experiment the concentration of HBV core chains was maintained at 50% of the standard conentration, and thus failed to rise to the necessary threshold for assembly. Thus, under the appropriate conditions, unassembled chains appear to be competent to form mature capsids.

### Example 18

### Completed Capsids Can Be Released By Manipulation of the Isolated Pellet

Having found an association of CC 60 with multimeric complexes, we wished to determine whether any of these complexes constitute intermediates in the assembly of the final capsid product and whether energy substrates play a role in the progression of such intermediates. Molecular chaperones are known to be involved in solubilizing aggregates of misfolded protein as well as in facilitating correct folding and assembly of polypeptides as discussed above. Thus, CC 60 could be associated with multimeric complexes in the pellet and middle fractions either because these complexes represent “dead end pathways” consisting of aggregates of misfolded or misassembled protein, or because these complexes represent productive intermediates along the pathway towards assembly of completed capsids. To address this, pellet material was isolated by fractionating the products of a 30-min translation of HBV core on a sucrose gradient and resuspending the pellet in buffer. The resuspended pellet was divided into equal aliquots and treated either with aphyase or with buffer for 90 min at 24° C. Radiolabeled material from the pellet chased to the middle with aphyrase treatment (FIG. 23A, top), but not with incubation in buffer (FIG. 23A, bottom). When fractions 6 and 7 were collected after apyrase treatment and centrifuged to equilibrium on a CsC1 gradient, most of the radiolabeled material was found to comigrate with authentic core particles (data not shown). Thus, apyrase treatment of isolated pellet material results in release of completed capsids from the pellet.

When the isolated pellet was treated with the energy mix used in cell-free translations (containing ATP, GTP, and creatine phosphate) along with the wheat germ extract, radiolabeled core polypeptides in the pellet were found to chase into both middle and top fractions (FIG. 23B, top). Once again, when the radiolabeled material in the middle was examined by equilibrium sedimentation, a small portion had a buyoant density identical to that of authentic capsids (data not shown). Treatment of the isolated pellet with either wheat germ extract or energy mix alone resulted in chase of a much smaller amount of radiolabeled material to the middle of the gradient (data not shown). Treatment of the isolated pellet with apyrase and wheat germ extract (FIG. 23B, bottom). Produced the same result as treatment with apyrase along (FIG. 23A, top). Thus, the addition of energy substrates results in release of both unassembled core polypeptides as well as assembled capsids from the pellet. Additional data demonstrated that the polysomes do not play a role in the pellet: (a) the protein synthesis inhibitor emetine did not affect the results of treatment of the isolated pellet with energy substrates or apyrase; ad (b) as previously mentioned, treatment of translation products with 10 mM EDTA had no effect on relative distribution of labeled core polypeptides in the top, middle, and pellet regions of the gradients (data not shown). The ability of the pellet to chase into completed capsids with various manipulations of energy substrates indicates that some of the material in the pellet constitutes an intermediate in the pathway to completed capsids.

### Example 19

### Preparation of Library that Cross-References Viral Families and Host Cell Proteins

A library that cross-references host cell proteins and particular viral families is developed by preparing capsids for at least one member of each viral family using the cell-free system described in Example 1 and using velocity sedimentation, separating out the capsid assembly intermediates that are formed. Antibodies raised against known host chaperones are then used to screen the assembly intermediates. Examples of such chaperones include TCP-1, HP68 and CC 60.

TABLE 2
Characteristics of Host Proteins

| | | | | Assembly |
|---|---|---|---|---|
| | | | | intermediate |
| | | | | (and capsid) |
| | Host | | Capsid | sedimentation |
| Virus | Protein | Antibody | protein(s) | coefficients |
| Lentivirus | HP68 | TCP-1 (23c) | Gag, pol, | 10S, 80S, 150S, |
| (HIV-1, | | | vif | 500S (750S) |
| HIV-2, SIV) | | | | |
| HCV | | TCP-1 (60-C) | | 10-20S, large |
| | | | | (100S) |
| HBV | CC 60 | TCP-1 (60-N) | | 20S, large (100S) |
| | hsp90 | TF55 | | |
| M-MPV | | TCP-1 | | |

One universal step in the lifecycle of all viruses is formation of the capsid. As the above results show, for multiple viruses from different families, capsid assembly is not spontaneous but rather is catalysed by the action of host proteins and occurs via assembly intermediates. An obligate, stereotyped, pathway of capsid assembly, distinct in both host factors and assembly intermediates for each different class of viruses studied to date, occurs. The cell-free transflation system in which these discoveries were made allows deconstruction of any virus by determination of which host proteins the virus utilizes without regard to conditions necessary to propagate or grow the virus per se. Furthermore in that system the assembly intermediates can be detected and enriched. Both the host proteins and the assembly intermediates are promising candidate anti-viral targets, as evidence demonstrates in one case that expression of a dominant negative mutant of one such host protein terminates release of virus from infected cells. Thus, anti-capsid therapy, in the form of small molecule drugs that interfere with those host proteins or the flux of intermediates involved in capsid assembly, are a promising new line of rapid responses to a viral threat, that may prove effective even before the virus has been identified and/or the ability to culture the virus has been established. This step of capsid assembly has not previously been the target of antiviral therapy because it had been believed that the capsid was formed spontaneously by “self-assembly” and therefore lacked a specific protein target.

All references cited herein are incorporated herein by reference, as if set forth in their entirety.

Although the foregoing invention has been described in some detail by way of illustration and example for purposes of clarity of understanding, it will be obvious that certain changes and modifications may be practiced within the scope of the appended claims.

What is claimed is:

1. A method for identifying, in a non naturally-occurring viral genome, a viral gene required for capsid assembly, said method comprising:
isolating nucleic acid putatively encoding said viral gene from said viral genome;
programming a cell free translation system with said nucleic acid; and
determining that capsid assembly has occurred as an indication that said viral gene is required for capsid assembly.

2. A composition comprising:
isolated nucleic acid from a non-naturally occurring viral genome encoding a viral gene required for capsid assembly.

3. A method for identifying a compound that inhibits capsid assembly of a non naturally-occurring virus, said method comprising:
programming a cell free translation system with nucleic acid encoding a protein required for capsid assembly of said non naturally-occurring virus in the presence and absence of said compound; and
determining whether capsid assembly has occurred as an indication as to whether said compound inhibits capsid assembly, wherein inhibition of capsid assembly is inferred from a change selected from the group consisting of (a) a change in distribution of assembly intermediates in the cell-free system; (b) a change in localization of host proteins in the cell-free system on glycerol or sucrose gradients; (c) a change in distribution of assembly intermediates in cells; (d) a change in level of assembly intermediates produced in cells; and (e) a change in co-localization of host protein and capsid protein in cells as observed during viral infection.

4. A composition comprising:
a compound identified according to the method of claim 3, wherein capsid assembly is inhibited in the presence of said compound.

5. A method for obtaining one or more host proteins that interact with one or more viral proteins required for capsid assembly of a non naturally-occurring virus, said method comprising:
programming a cell free translation system with nucleic acid encoding one or more protein required for capsid assembly of said non naturally-occurring virus whereby translation products for said one or more capsid proteins are produced;
incubating said translation mixture for a period of time sufficient for said translation products to assemble into one or more capsid intermediates, wherein said one or more capsid intermediates each comprise a complex of polymerized viral capsid protein and a host protein;
isolating said one or more capsid intermediates; and
dissassociating said one or more capsid intermediates whereby said one or more host proteins are obtained.

6. A capsid intermediate comprising a host protein obtained according to the method of claim 5.

7. A host protein obtained according to the method of claim 5.

8. A human homologue of a host protein obtained according to the method of claim 7.

9. Antibodies to a host protein according to claim 7.

10. The antibodies according to claim 9, wherein said antibodies inhibit binding of a host protein to one or more viral proteins required for capsid assembly of a non naturally-occurring virus.

11. A method of obtaining a capsid intermediate involved in assembly of a non naturally-occurring virus, said method comprising:
combining a nucleic acid enoding a viral gene required for capsid assembly with a cell-free protein translation mixture;
incubating said mixture for a period of time sufficient to assemble translation products of said viral gene into viral capsid intermediates;
separating said translation mixture into fractions of one or more capsid intermediates; and
isolating said one or more capsid intermediates whereby capsid intermediates of said non-naturally occurring virus are obtained.

12. A method of identifying host proteins involved in capsid assembly of a non naturally-occurring virus, said method comprising:
denaturing host proteins obtained according to the method of claim 5;
sequencing-said individual host proteins, and
comparing the sequences of said individual host proteins to known sequences of host proteins, whereby the identity of host proteins that are involved in capsid assembly of said non naturally-occurring virus are obtained.

13. A method for identifying compounds that interfere with capsid assembly of a non naturally-occurring virus, said method comprising:
expressing a protein required in said capsid assembly in a mammalian cell;
identifying co-localization of said protein and one or more host protein using immunofluorescence in said mammalian cells; and
screening compounds for those that interfere with co-localization of said protein required in said capsid and said one or more host protein in said mammalian cells, whereby compounds that interfere with capsid assembly are identified by a change from co-localization of said immunofluorescence to a diffuse staining pattern.

14. The method according to claim 13, wherein said compounds do not cause toxcicity or upregulate host stress proteins in said mammalian cells.

15. A method of identifying a compound that inhibits capsid assembly of a non naturally-occurring virus, said method comprising:
adding a test compound to a cell-free translation mixture programmed with nucleic acid from virus encoding one or more protein required for capsid assembly whereby capsids are produced;
comparing assembly in the absence of said test compound to assembly in the presence of said test compound, wherein less assembly measured in the presence of said compound is indicative of a compound that inhibits capsid assembly.

16. The method according to claim 15, wherein said compound is a small molecule.

17. A method of inhibiting capsid formation in a cell of a non naturally-occurring virus, said method comprising:
providing to said cell a compound selected according to the method of Claim 15.

18. The method according to claim 17, wherein said cell is a human cell.

19. The method according to claim 15, wherein said compound is an anti-capsid antibody.

20. A method for treating symptoms, in an animal in need thereof, of a bioterrorist attack with an unknown viral agent, said method comprising:
isolating nucleic acid putatively encoding a viral gene involved in capsid assembly from said viral genome;
programming a cell free translation system with said nucleic acid for a period of time sufficient to assemble translation products of said viral gene into viral capsid intermediates;
separating said translation products into fractions of one or more capsid intermediates;
isolating said one or more capsid intermediates;
separating said capsid intermediates to obtain one or more host proteins that were bound to one or more viral protein in said capsid intermediates;
comparing a biochemical characteristic of said individual one or more host proteins to a library comprising biochemical characteristics of a plurality of viral capsid assembly chaperones individually cross-referenced with one or more small molecules that inhibit interaction between an individual member of said library and a viral capsid protein; and
providing said animal with a small molecule cross-referenced with an individual member of said library having a biochemical characteristic in common with said one or more host protein, whereby said symptoms are treated.

21. The method according to claim 20, wherein said biochemical characteristic is an amino acid sequence of a binding region for a viral capsid protein.

22. The method according to claim 21, wherein said animal is a mammal.

23. The method according to claim 21, wherein said mammal is a human.

24. A method for producing viral capsids of a non-naturally occurring virus, said method comprising:
programming a cell free translation system with said nucleic acid from said virus; and
incubating said translation mixture for a period of time sufficient for said translation products to assemble into capsids; and
isolating said capsids.

25. Antibodies to viral capsids of a non-naturally occurring virus.

26. The antibodies according to claim 25 wherein said antibodies are monoclonal antibodies.
